(19) United States
(12) Patent Application Publication
(10) Pub. No.: US 2004/0072368 A1
(43) Pub. Date: Apr. 15, 2004

(54) Lipoprotein receptor

(76) Inventors: Laurent Martinez, Toulouse cedex 3 (FR); Sebastien Jacquet, Toulouse cedex 3 (FR); Corinne Rolland, Toulouse cedex 3 (FR); Francois Terce, Toulouse cedex 3 (FR); Xavier Collet, Toulouse cedex 3 (FR); Bertrand Perret, Toulouse cedex 3 (FR); Ronald Barbaras, Toulouse cedex 3 (FR); Eric Champagne, Toulouse cedex 3 (FR); Jean-Pierre Esteve, Toulouse cedex 3 (FR); John Walker, Cambridge (GB); Michael Runswick, Cambridge (GB)

Correspondence Address: PALMER & DODGE, LLP, KATHLEEN M. WILLIAMS, 111 HUNTINGTON AVENUE, BOSTON, MA, 02199, US

(21) Appl. No.: 10/242,686

(22) Filed: Sep. 12, 2002

(51) Int. Cl. G01N 33/53; G01N 33/00; G01N 33/543

(52) U.S. Cl. 436/518; 435/7.1

(57) ABSTRACT

This invention relates to the use of at least one domain of ATP synthase as a lipoprotein receptor.

### FIELD OF INVENTION

The present invention relates to the use of at least one domain of ATP synthase as a lipoprotein receptor and to methods for identifying a lipoprotein receptor.

Moreover, the present invention relates to assay methods, processes, pharmaceutical compositions and agents that are useful in the treatment and/or prevention of a disease such as cardiovascular disease.

### BACKGROUND TO THE INVENTION

Cardiovascular diseases such as coronary heart disease and stroke have increasingly become a major cause of deaths. It has been reported that an elevated plasma cholesterol level—such as in cholesterolemia—causes the deposition of fat, macrophages and foam cells on the wall of blood vessels, which leads to atherosclerosis (19). Both elevated plasma cholesterol levels and atherosclerosis are strongly associated with cardiovascular and other diseases. There is therefore a need to control the levels of cholesterol in the blood.

Cholesterol and other water-insoluble lipids are transported in the bloodstream on lipoprotein particles. These particles consist of an amphipathic shell of phospholipid and apolipoprotein surrounding a non-polar core of triglyceride and cholesterol. The major cholesterol transporter in human blood is low density lipoprotein (LDL), thus high blood cholesterol is synonymous with an excess of LDL particles in the bloodstream. LDL is produced first as a precursor particle, very low density lipoprotein (VLDL). VLDL carries a large core of triglyceride. While in the circulation, the triglyceride is hydrolysed by lipoprotein lipase, an enzyme that resides on the luminal surface of the capillary endothelium of cells that will either store (adipocytes) or oxidize (muscle) the fatty acids that are released by the lipolysis reaction. Upon depletion of most of its triglyceride core, the remaining particle becomes LDL. Thus, through selective removal of its triglyceride core, a triglyceride-rich lipoprotein becomes a cholesterol-rich lipoprotein.

LDL is the major cholesterol transporter in human blood, thus high blood cholesterol is synonymous with a high LDL level. LDL is cleared from the bloodstream primarily through LDL receptors. Mutations in the LDL receptor are a common cause of hypercholesterolemia. The transcription of the LDL receptor gene is regulated by an unusual transcription factor that is membrane bound. It is released and thereby activated by proteolytic cleavage, a process inhibited by sterols. In this manner, sterols inhibit the transcription of the LDL receptor and other sterol-responsive genes.

LDL is scavenged from the blood stream by receptors located on the cell membranes. Descriptions of the LDL receptor, its structure, and its general functionality in interaction with other cellular processes can be found in Schneider, Bio. Et. Biophys. Acta., 988, pages 307-317 (1989) and Hobbs, et al., Annu. Rev. Genet., 24, pages 133-170 (1990). The LDL receptor protein is a cell surface glycoprotein that regulates plasma cholesterol by mediating endocytosis of lipoproteins. The human LDL receptor is a protein of 860 amino acids encoded by a gene which actuates the transcription of an mRNA of 5.3 kb in length. The mRNA of the human LDL receptor gene includes a long 3′ untranslated region, as well as a signal peptide which actuates transport of the protein to the plasma membrane, and which is cleaved from the mature form of the protein.

Another type of lipoprotein is high-density lipoprotein (HDL), which unlike LDL, is beneficial to health. A recent study showed that an increase in the plasma HDL level is inversely related to the occurrence of heart disease (20) and so a low plasma HDL level is an important risk factor of atherosclerosis (21). In addition, it has been have verified that plasma HDL has anti-inflammatory and anti-atherosclerosis activities (22). Thus, the risk of developing atherosclerosis, the leading cause of mortality in industrialised countries, is inversely related to the plasma concentrations of HDL-cholesterol. This protective effect of HDL is attributed to the major role of HDL in a process called “reverse cholesterol transport”, a process by which excess cholesterol is extracted from peripheral cells by HDL and delivered to the liver for its elimination. Reverse cholesterol transport, therefore, reduces cholesterol accumulation in the artery wall (Reichl, D and Miller, N. E., Arteriosclerosis 9, 785 (1989)). Because there is no cholesterol accumulation in extrahepatic organs, cholesterol must be transported to the liver by HDL for ultimate excretion into bile, either as free cholesterol, or as bile acids that are formed from cholesterol (Kwiterovich, P. O., Amer. J Cardiol. 82, 13Q, (1998)). The major functional role of HDL is to remove cholesterol from peripheral tissues including atherosclerotic lesions and taking cholesterol in its ester form to the liver for elimination. In this process, HDL particles mediate the efflux and the transport of cholesterol from peripheral cells to the liver, for further metabolism and bile excretion. The physiological importance of this pathway has prompted numerous studies focused on the identification of cell surface receptors for HDL, which might regulate reverse cholesterol transport.

Binding sites for HDL, or its major apolipoprotein (apoA-I), have been identified in liver plasma membranes (1) and human hepatoma cells (2). Both, a high-affinity (10-9M) binding site and another component of lower affinity (10-7M) were evidenced, the latter possibly reflecting weaker protein-protein and/or protein-lipid interactions. Interestingly, lipid-free apoA-I (named free-apoA-I) binds only to the high-affinity sites and thus constitutes a selective ligand to study this receptor. It has been previously been shown that HDL is internalised in HepG2 cells, via the formation of clathrin-coated vesicles, following engagement of the low affinity binding sites (3). It has been further observed that binding and endocytosis of HDL are modulated by metabolic events that affect HDL structure and distribution. In the liver, large-sized HDL particles, enriched in triglycerides (triglyceride rich HDL₂ or TG-HDL₂), are a preferential substrate for hepatic lipase, acting at the endothelial surface of sinusoid capillaries and leading to the formation of a triglyceride and phospholipid-poor “remnant-HDL” (4). TGHDL₂ displays only low-affinity binding whereas the post-lipolysis remnant-HDL could bind to both low and high-affinity sites. Moreover, the remnant-HDL is internalised faster and in higher amounts than their parent TG-HDL₂ (4).

Hammad et al. (1999) Proc. Natl. Acad. Sci. 96 p10158-10163 have identified an HDL receptor identified as cubulin which may be involved in the embryonic acquisition of maternal HDL and renal catabolism of filterable forms of HDL.

Beisiegel and Mahley (13) have identified apolipoprotein E-binding proteins comprising the α—and β-subunits of ATP synthase. However, the physiological role for these proteins in ApoE metabolism was not determined.

ATP synthase expressed on the cell surface and acting as a ligand receptor has been reported for lymphocytes (10) and human endothelial cells (11, 12). In the later case, cell surface ATP synthase was acting as a receptor for different ligands like angiostatin.

An improved understanding of lipoprotein catabolism, for example, HDL catabolism, would open up new perspectives in the control of cholesterolemia, which is a major issue in cardiovascular disease research.

### SUMMARY OF THE INVENTION

The present invention is based upon the surprising finding that at least one domain of ATP synthase may be used as a lipoprotein receptor, for example, an HDL or a lipid free-apolipoprotein A-I (free-apoA-I) receptor, and is a major partner in the regulation of cholesterol homeostasis.

It is desirable to improve the functionality of HDL by acting on proteins and receptors involved in reverse cholesterol transport in such a way as to increase the half life of lipoproteins—such as apoAI-HDL—and/or to increase the delivery of cholesteryl esters to the liver.

Thus, in a first aspect, the present invention relates to the use of at least one domain of ATP synthase as a lipoprotein receptor.

Preferably, binding of a lipoprotein to the lipoprotein receptor stimulates lipoprotein endocytosis.

Preferably, binding of a lipoprotein to the lipoprotein receptor stimulates HDL endocytosis.

Preferably, binding of a lipoprotein to the lipoprotein receptor stimulates holo-HDL endocytosis.

Preferably, the lipoprotein receptor is a high density lipoprotein (HDL) receptor. More preferably, the lipoprotein receptor is lipid free-apolipoprotein A-I (free-apoA-I) receptor.

Preferably, at least one domain of ATP synthase comprises one or more subunits of the F₁ domain of ATP synthase. More preferably, at least one domain of ATP synthase comprises the beta-subunit of the F₁ domain of ATP synthase.

Preferably, the beta-subunit of the F₁ domain of ATP synthase comprises SEQ ID No.1.

Preferably, the beta-subunit of the F₁ domain of ATP synthase comprises a polypeptide encoded by SEQ ID No.2.

Preferably, at least one domain of ATP synthase is present on the surface of a cell. Preferably, the cell is a hepatocyte. More preferably, the cell is a primary human hepatocyte, an immortalised human hepatocyte or a HepG₂ cell.

In a second aspect, the present invention relates to a method for identifying a lipoprotein receptor comprising the steps of: (a) contacting a sample with lipoprotein; (b) obtaining one or more lipoprotein bound proteins; and (c) determining if the lipoprotein bound proteins comprise at least one domain of ATP synthase.

Preferably, the lipoprotein is high density lipoprotein (HDL). More preferably, the lipoprotein is lipid free-apolipoprotein A-I (free-apoA-I).

Preferably, at least one domain of ATP synthase comprises one or more subunits of the F₁ domain of ATP synthase.

Preferably, at least one domain of ATP synthase comprises the beta-subunit of the F₁ domain of ATP synthase.

Preferably, the beta-subunit of the F₁ domain of ATP synthase comprises SEQ ID No.1.

Preferably, the beta-subunit of the F₁ domain of ATP synthase comprises a polypeptide encoded by SEQ ID No.2.

Preferably, the sample comprises solubilised membranes. More preferably, the solubilised membranes are solubilised liver plasma membranes.

Preferably, the sample is contacted with immobilised lipoprotein. More preferably, the sample is contacted with immobilised lipoprotein using surface plasmon resonance or affinity chromatography.

The method according to the second aspect of the present invention may comprise the additional step (d) of determining if at least one domain of ATP synthase is localised at the surface of a cell.

Preferably, step (d) is performed using immunofluorescence microscopy or fluorescence assisted flow cytometry.

Preferably, immunofluorescence microscopy or fluorescence assisted flow cytometry are performed with an anti-ATP synthase monoclonal antibody. More preferably, the monoclonal antibody is an anti-β-subunit ATP synthase monoclonal antibody.

In a third aspect, the present invention relates to an assay method comprising the steps of: (a) identifying one or more agents that modulate ATP hydrolysis; and (b) determining if the one or more agents modulate the activity of a lipoprotein receptor.

Preferably, the lipoprotein receptor modulates lipoprotein endocytosis.

Preferably, the lipoprotein receptor stimulates HDL endocytosis.

Preferably, the lipoprotein receptor stimulates holo-HDL endocytosis.

Preferably, ATP is hydrolysed by at least one domain of ATP synthase. More preferably, at least one domain of ATP synthase comprises one or more subunits of the F₁ domain of ATP synthase. Most preferably, at least one domain of ATP synthase comprises the beta-subunit of the F₁ domain of ATP synthase.

Preferably, the beta-subunit of the F₁ domain of ATP synthase comprises SEQ ID No.1.

Preferably, the beta-subunit of the F₁ domain of ATP synthase comprises a polypeptide encoded by SEQ ID No.2.

Preferably, at least one domain of ATP synthase is present on the surface of a cell.

Preferably, the lipoprotein receptor is a high density lipoprotein (HDL) receptor. More preferably, the lipoprotein receptor is a lipid free-apolipoprotein A-I (free-apoA-I) receptor.

Preferably, the agents modulate the activity of a further entity.

Preferably, the agent is an antagonist that decreases lipoprotein endocytosis.

Preferably, the agent is an agonist that increases lipoprotein endocytosis. More preferably, the agent is an antagonist of IF1.

Preferably, the assay method is used to screen for agents that are useful in the treatment and/or prevention of disease.

In a fourth aspect, the present invention relates to the use of at least one domain of ATP synthase in an assay method to identify one or more agents that that are useful in the treatment and/or prevention of disease.

In a fifth aspect, the present invention relates to the use of at least one domain of ATP synthase in an assay method to identify one or more agents that modulate lipoprotein endocytosis.

In a sixth aspect, the present invention relates to a process comprising the steps of: (i) performing the assay method according to the third aspect of the present invention; (ii) identifying an agent capable of modulating lipoprotein endocytosis; and (iii) preparing a quantity of that agent.

In a seventh aspect, the present invention relates to a process comprising the steps of: (i) performing the assay method according to the third aspect of the present invention; (ii) identifying an agent capable of modulating lipoprotein endocytosis; (iii) preparing a quantity of that agent; and (iv) preparing a pharmaceutical composition comprising that agent.

In an eighth aspect, the present invention relates to a process comprising the steps of: (i) performing the assay method according to the third aspect of the present invention; (ii) identifying an agent capable of modulating lipoprotein endocytosis; (iii) modifying said agent; and (iv) preparing a pharmaceutical composition comprising said modified agent.

In a ninth aspect, the present invention relates to a pharmaceutical composition comprising an agent identified by the assay method of the third aspect of the present invention or the process of the sixth, seventh or eighth aspects of the present invention admixed with a pharmaceutically acceptable carrier, diluent, excipient or adjuvant and/or combinations thereof.

In a tenth aspect, the present invention relates to a process of preparing a pharmaceutical composition comprising admixing an agent identified by the assay method of the third aspect of the present invention or the process of the sixth, seventh or eighth aspects of the present invention with a pharmaceutically acceptable diluent, carrier, excipient or adjuvant and/or combinations thereof.

In an eleventh aspect, the present invention relates to the use of at least one domain of ATP synthase in the manufacture of a pharmaceutical composition for the treatment and/or prevention of a disease.

In a twelfth aspect, the present invention relates to a method of treating a disease in a human or animal which method comprises administering to an individual an effective amount of a pharmaceutical composition comprising an agent identified by the assay method of the third aspect of the present invention or the process of the sixth, seventh or eighth aspects of the present invention, wherein the agent is capable of modulating the disease and wherein said composition is optionally admixed with a pharmaceutically acceptable carrier, diluent excipient or adjuvant and/or combinations thereof.

Preferably, said one or more agents are formulated into one or more compositions for use in medicine.

In a thirteenth aspect, the present invention relates to an agent identifiable, preferably identified, by the assay method according to the third aspect of the present invention.

In a fourteenth aspect, the present invention relates to an agent identifiable, preferably identified, by the assay method according to the third aspect of the present invention for use in the treatment and/or prevention of disease.

Preferably, the disease is selected from: cardiovascular disease, coronary heart disease, stroke, pancreatitis, atherosclerosis, gout, and/or type 2 diabetes.

### DETAILED DESCRIPTION OF THE INVENTION

ATP Synthase

The mitochondrial electron transport (or respiratory) chain is a series of enzyme complexes in the mitochondrial membrane that is responsible for the transport of electrons from NADH to oxygen and the coupling of this oxidation to the synthesis of ATP (oxidative phosphorylation). ATP then provides the primary source of energy for driving a cell's many energy-requiring reactions.

ATP synthase is the enzyme complex at the terminus of this chain and serves as a reversible coupling device that interconverts the energies of an electrochemical proton gradient across the mitochondrial membrane into either the synthesis or hydrolysis of ATP. This gradient is produced by other enzymes of the respiratory chain in the course of electron transport from NADH to oxygen. When the cell's energy demands are high, electron transport from NADH to oxygen generates an electrochemical gradient across the mitochondrial membrane. Proton translocation from the outer to the inner side of the membrane drives the synthesis of ATP. Under conditions of low energy requirements and when there is an excess of ATP present, this electrochemical gradient is reversed and ATP synthase hydrolyses ATP. The energy of hydrolysis is used to pump protons out of the mitochondrial matrix.

The mammalian ATP synthase complex consists of sixteen different polypeptide subunits (Walker, J. E. and Collinson, T. R. (1994) FEBS Lett. 346 39-43). Ten polypeptides (subunits a, b, c, d, e, f, g, F6, OSCP, and A6L) comprise the proton-translocating, membrane spanning F₀ portion of the complex. Six of these polypeptides (subunits alpha, beta, gamma, delta, epsilon and an ATPase inhibitor protein, IF₁) comprise the globular catalytic F₁ domain of the ATPase portion of the complex. Preferably, the receptor according to the present invention comprises one or more domains of ATP synthase at the cell surface.

More preferably, at least one domain of ATP synthase comprises one or more subunits of the F₁ domain of ATP synthase.

F₁ is the catalytic part of ATP synthase, which projects inward from inner mitochondrial membranes. The quaternary structure of F₁ consists of 3 each of alternating alpha and beta subunits forming a cylindrical complex attached to the membrane-embedded F₀ proton channel. The central cavity of the cylinder is occupied by the alpha-helical C-terminal domain of the gamma subunit. When hydrogen ions flow through the membrane via the disc of c subunits in the Fo part, the disc imparts a twist to the γ-subunit, which protrudes from the F₁ part and is attached to the disc. The three alpha and three beta subunits in the F₁ part cannot rotate, however. They are locked in a fixed position by the β subunit, which in turn is anchored in the membrane. Thus the γ subunit rotates inside the cylinder formed by the six alpha and beta subunits. Since the gamma subunit is asymmetrical it compels the beta subunits to undergo structural changes. This leads to the beta subunits binding ATP and ADP with differing strengths. The interconversion of these states, and hence the continuous production of ATP, occurs as the γ subunit rotates.

Members of the F₁F₀ family of ATP synthases are present in bacteria, in chloroplast membranes, and in mitochondria. [Molecular Biology of the Cell, Alberts et al., eds. Garland Publishing, Inc., New York (1983), pages 484-510.] The enzyme is well conserved; the β subunit polypeptides from different sources show exceptionally strong sequence homology (almost 50% sequence identity), while the minor F₁ subunit polypeptides show more sequence and size variation. In fact, in the highly conserved regions of the beta subunit, the primary amino acid sequences were identical among tobacco, spinach, maize, bovine, E. coli and S. cerevisiae (Takeda et al., J Biol. Chem., 260(29):15458-15465 (1985)).

Most preferably, at least one domain of ATP synthase comprises the beta-subunit of the F₁ domain of ATP synthase.

The beta subunit of mitochondrial ATP synthase is encoded by a nuclear gene and assembled with the other subunits encoded by both mitochondrial and nuclear genes. The enzyme catalyses ATP formation, using the energy of proton flux through the inner membrane during oxidative phosphorylation. Two subunits are encoded by a mitochondrial gene and the others by a nuclear gene. The numbers of mitochondria per cell vary greatly depending on the developmental stage, cell activity, and type of tissue. The molecular mechanism for co-ordinating the 2 genetic systems is unknown. Ohta et al. (1988) J. Biol. Chem. 263: 11257-11262, cloned cDNA of the human beta subunit. The gene contains 10 exons, with the first exon corresponding to the noncoding region and most of the presequence which targets this protein to the mitochondria. Neckelmann et al. (1989) Cytogenet. Cell Genet. 51 1051 sequenced the human ATP synthase beta-subunit gene and demonstrated that it is preferentially expressed in heart and skeletal muscle. The gene was found to have 10 exons encoding a leader peptide of 49 amino acids and a mature protein of 480 amino acids. Kudoh et al. (1989) Cytogenet. Cell Genet. 51: 1026 assigned the ATPMB locus to the p13-qter region of human chromosome 12 by analysis of human-mouse somatic cell hybrid DNA and by use of flow-sorted chromosomes. They assigned 2 related sequences, ATPMBL1 and ATPMBL2, to chromosome 2 and 17, respectively.

Preferably, the beta-subunit of the F₁ domain of ATP synthase comprises SEQ ID No.1 or a variant, derivative or homologue thereof.

Preferably, the beta-subunit of the F₁ domain of ATP synthase comprises a polypeptide encoded by SEQ ID No.2 or a variant, derivative or homologue thereof.

The F₁ domain of the ATP synthase of the lipoprotein receptor of the present invention may even comprise the β-subunit associated with the α-subunit.

Lipoprotein Receptor

As used herein, the term “lipoprotein receptor” refers to any receptor that is capable of binding conjugated, water soluble proteins in which the non-protein moiety consists of one or more lipids. The lipid may be triacylglycerol, cholesterol, or phospholipid, or a combination of these.

Preferably, the lipoprotein is cholesterol—such as very low density lipoprotein (VLDL) cholesterol, intermediate density lipoprotein (IDL) cholesterol, low density lipoprotein (LDL) cholesterol, or high density lipoprotein (HDL) cholesterol. A fifth class of lipoprotein is chylomicrons, which occur only after feeding.

Several methods are available for the determination of lipoprotein in plasma and serum (Mills, G. L., Lane, P. A., Weech, P. K.: A guidebook to lipoprotein technique. Elsevier, Amsterdam, 1984; Cremer, P. and Seidel, D.: Dtsch. Gesell. Klin. Chem. Mittl. 21, 1990, 215-232).

Several other methods for the determination of lipoprotein may also be used. By way of example, Japanese Patent Application No. 7-301636 discloses a method for exclusively measuring HDL cholesterol by use of a surfactant and a sugar compound. Japanese Patent Application No. 6-242110 discloses a method for exclusively measuring cholesterol in a lipoprotein by agglutinating lipoproteins other than the lipoprotein to be measured. Methods for measuring LDL cholesterol include a method in which LDL is separated from other lipoproteins by ultracentrifugation to measure cholesterol and a method in which lipid is stained after separation through electrophoresis so as to measure the intensity of the developed colour. Other methods are disclosed in U.S. Pat. No. 6,333,166 and U.S. Pat. No. 5,925,534.

Preferably, the lipoprotein is HDL.

HDL circulates in the bloodstream, extracting cholesterol from body tissues and transporting it to the liver for excretion or recycling. Nascent HDL particles are discoidal, consisting of a phosphatidylcholine bilayer and a protein shell which shields the hydrophobic lipid tails from the aqueous environment. As it circulates in the body, HDL collects cholesterol, which is then stored in the lipid bilayer. Increased efficiency is achieved though the activation of the lecithin-cholesterol acyl transferase (LCAT) enzyme which converts the amphipathic cholesterol stored in the bilayer into hydrophobic cholesterol esters which collect among the lipid tails. This induces a transformation of the HDL disk to a spherical form in which a hydrophobic core of cholesterol esters is shielded by a combination of lipid and protein. At this stage cholesterol collection ceases and the mature HDL particle are recognised by the liver.

More preferably, the lipoprotein is free-apoA-I.

Free-apoA-I is the major apoprotein of HDL and is a relatively abundant plasma protein. Apolipoprotein A1 (apo A1) is the main protein constituent of HDL and is the primary acceptor for cholesterol in HDL. Therefore, HDL is responsible for most of the reverse cholesterol transport in man, as it is the only particle capable of receiving cholesterol from the peripheral cells.

Free-apoA-I is a single polypeptide chain with 243 amino acid residues of known primary amino acid sequence (Brewer et al. (1978) Biochem. Biophys. Res. Commun. 80: 623-630). ApoA-I is a cofactor for LCAT, which is responsible for the formation of most cholesteryl esters in plasma. ApoA-I also promotes efflux of cholesterol from cells. The liver and small intestine are the sites of synthesis of apoA-I. The primary translation product of the apoA I gene contains both a pre and a pro segment, and posttranslational processing of apoA-I may be involved in the formation of the functional plasma apoA-I isoproteins.

Lipoprotein Endocytosis

In a preferred embodiment of the present invention, binding of a lipoprotein to the lipoprotein receptor of the present invention stimulates lipoprotein endocytosis i.e. the lipoprotein receptor is involved in the internalisation of lipoprotein into cells, for example, hepatocytes.

Thus, as described herein, a unique effect for a cell surface ATP synthase is demonstrated, since ATP synthase activity generates a major modulation of lipoprotein endocytosis, for example, HDL—such as holo HDL (protein and lipid) and TG-HDL₂ endocytosis.

Various methods may be used to determine the functional activity of the lipoprotein receptor of the present invention. By way of example, cells—such as hepatocyte cells may be incubated with labelled ADP to detect ATP synthesis activity, or with labelled ATP, to measure ATP hydrolytic activity, in the presence or absence of lipoprotein—such as free-apoA-I. The different nucleotides generated in cell culture medium may be identified by Thin Layer Chromatography (TLC) and HPLC techniques, the latter allowing the precise quantification of the nucleotides.

Purified IF₁ protein, the natural inhibitor protein of mitochondrial F₁-ATPase, interacts with the β-subunit to inhibit the hydrolytic activity of the ATP synthase (14). Purified IF₁ protein may be used to determine if a decrease in the radiolabelled ADP generated occurs. By way of example, if the addition of F₁-ATPase inhibits the hydrolytic activity of ATP synthase then this suggests that the ATP synthase may function as an ATP hydrolase.

Without wishing to be bound by theory, HDL endocytosis may be ADP-dependent. The ATP synthase present on the surface of a cell—such as a hepatocyte, may hydrolyse extracellular ATP to ADP, which in turn activates HDL endocytosis. This mechanism may be stimulated by the high affinity binding of apoA-I to the β-subunit of ATP synthase, inducing an overproduction of ADP and increasing HDL endocytosis.

Cell Surface Localisation

In a preferred embodiment, at least one domain of ATP synthase that is used as a lipoprotein receptor is present on the surface of a cell.

Preferably, the cell is a hepatocyte. More preferably, the cell is a primary human hepatocyte, an immortalised human hepatocyte or a HepG₂ cell.

Various methods may be used to determine if at least one domain of ATP synthase that is used as a lipoprotein receptor is localised at the cell surface.

By way of example, immunofluorescence microscopy with a monoclonal antibody may be used.

The cell to be analysed is fixed on a slide and/or permeabilised and the protein, for example, the β subunit of the F₁ domain of ATP synthase, is detected with a specific antibody, for example, a monoclonal antibody to a subunit of the F₁ domain of ATP synthase. The antibody detection technique may be indirect (ie. the slide is incubated first with an unconjugated specific antibody (primary antibody), followed by a fluorochromeconjugated antibody (secondary antibody). The antibody detection technique may be also be direct ie. the slide is incubated with a fluorochrome-conjugated antibody. Antibodies for immunofluorescent detection may be conjugated with various fluorochromes—such as fluorescein, rhodamine, phycoerythrin, or Texas Red.

By way of example, cells may be incubated with a primary antibody diluted in PBS (eg. mouse monoclonal IgG2a anti β-subunit of ATP synthase, mouse monoclonal IgG2a anti subunit 1 of cytochrome oxidase monoclonal or mouse IgG2a isotypic control). Immunostaining may be performed in the dark with anti-mouse alexa 488-conjugated IgG2a in staining buffer. For confocal microscopy, cells may be incubated with apo AI and then washed in PBS before fixation. Rabbit polyclonal anti apoA-I immunserum may be co-incubated with primary antibodies. Immunostaining may be performed with anti-mouse alexa 488-conjugated IgG2a and rhodamine-conjugated anti-rabbit IgG. The coverslips may be examined with a Zeiss Axioskop microscope or with a confocal microscope.

Cells may also be analysed by fluorescence-assisted flow cytometry and flow cytometry using a monoclonal antibody to a subunit of ATPase.

Flow cytometry is a powerful method for studying and purifying cells. It has found wide application, particularly in immunology and cell biology, however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%). For a general reference, see Flow Cytometry and Cell Sorting: A Laboratory Manual (1992) A. Radbruch (Ed.), Springer Laboratory, New York.

FACS machines collect fluorescence signals in one to several channels corresponding to different laser excitation and fluorescence emission wavelengths. Fluorescent labelling allows the investigation of many aspects of cell structure and function. The most widely used application is immunofluorescence: the staining of cells with antibodies conjugated to fluorescent dyes eg. fluorescein and phycoerythrin. This method is often used to label molecules on the cell surface, but antibodies can also be directed at targets within the cell. In direct immunofluorescence, an antibody to a particular molecule is directly conjugated to a fluorescent dye. Cells can then be stained in one step. In indirect immunofluorescence, the primary antibody is not labelled, but a second fluorescently conjugated antibody is added which is specific for the first antibody.

FACS may be performed directly, by labelling of a protein, or indirectly by using a reporter gene. Examples of reporter genes are β-galactosidase and Green Fluorescent Protein (GFP). β-galactosidase activity can be detected by FACS using fluorogenic substrates—such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells, which are excited by the different lasers and therefore assay two transfections at the same time. FACS machines and reagents for use in FACS are widely available from sources world-wide including Becton-Dickinson, or Arizona Research Laboratories (http://www.arl.arizona.edu/facs/).

Preferably, cells are labeled with a primary antibody. More preferably, the primary antibody binds at least one domain of ATP synthase. The cells are then washed and may be labeled with a secondary antibody—such as goat anti-rabbit IgG conjugated to fluorescein isothiocyanate.

Fluorescein (abbreviated by its commonly-used reactive isothiocyanate form, FITC) is currently the most commonly-used fluorescent dye for FACS analysis. FITC is a small organic molecule, and is typically conjugated to proteins via primary amines (i.e., lysines). Usually, between 3 and 6 FITC molecules are conjugated to each antibody; higher conjugations may result in solubility problems as well as internal quenching (and reduced brightness). Thus, an antibody will usually be conjugated in several parallel reactions to different amounts of FITC, and the resulting reagents will be compared for brightness (and background stickiness) to choose the optimal conjugation ratio. Fluorescein is typically excited by the 488 nm line of an argon laser, and emission is collected at 530 nm.

The cells are again washed, fixed in formalin and analysed. Dead/dying cells may be excluded from the analysis by selecting appropriate forward and side scatter populations.

Suitable imaging agents for use with FACS may be delivered to the cells by any suitable technique, including simple exposure thereto in cell culture, delivery of transiently expressing nucleic acids by viral or non-viral vector means, liposome-mediated transfer of nucleic acids or imaging agents, and the like.

By way of example, cells may be detached, pelleted and incubated in a buffer—such as PBS—with either mouse monoclonal antibodies (eg. monoclonal antibody to the human β-subunit of ATP synthase). Cells may then be washed in buffer and incubated with a secondary antibody—such as goat anti-rabbit IgG conjugated to fluorescein isothiocyanate. After a final wash, cells may be pelleted and resuspended in buffer. The mean relative fluorescence after excitation at a wavelength of 488 nm may be determined for each sample on a flow cytometer—such as a Coulter XL 4C flow cytometer and analysed with software—such as CELLQUEST (Becton-Dickenson).

Identifying a Lipoprotein Receptor

In a further aspect, the present invention relates to a method for identifying a lipoprotein receptor, for example, an HDL receptor, comprising the steps of: (a) contacting a sample with lipoprotein; (b) obtaining one or more lipoprotein bound proteins; and (c) determining if the lipoprotein bound proteins comprise at least one domain of ATP synthase.

As used herein, the term “sample” has its natural meaning. A sample may be any physical entity to be contacted with a lipoprotein

Preferably, the lipoprotein is HDL. More preferably, the lipoprotein is lipid free-apolipoprotein A-I (free-apoA-I).

The sample may be or may be derived from biological material—such as one or more cells, for example, hepatocytes.

Preferably, the sample comprises membrane proteins. Plasma membranes may be prepared using various methods in the art—such as by the aqueous two-phase partition procedure in which the dominant orientation is right-side-out (cytoplasmic side in) (3).

Specific enzymatic markers may be measured to confirm that the starting material comprises pure plasma membranes—such as pure hepatocyte plasma membranes (4).

More preferably, the sample comprises solubilised membrane proteins. More preferably, the solubilised membranes are solubilised liver plasma membranes—such as solubilised porcine liver plasma membranes.

Solubilisation of membrane proteins may be performed using various methods in the art. By way of example, the extraction process may comprise a number of different steps, for example: (1) removal of unbroken cells from the cell lysate by low speed centrifugation (20 min at 10 000 g); (2) isolation of the membrane particles from the supernatant by ultracentrifugation (60 min at >100,000 g); (3) washing of the membrane particle to remove all soluble proteins; and (4) solubilisation of protein from the membrane particles by a detergent. The extent of the solubilisation and the stability of the solubilised membrane protein depends on the detergent type and concentration. An important part of the solubilisation is the detergent-to-protein ratio. At low ratios, the membranes are lysed and large complexes are formed containing protein, detergent, and membrane lipids. With progressively larger ratios, smaller complexes are obtained. Finally, at ratios of 10:1 to 20:1 individual detergent-protein complexes are formed free of membrane lipids. To determine the optimal conditions it is important to vary both the detergent and the protein concentration. Commonly used detergents include Triton X-100, octylglucoside, CHAPS, Zwittergent 3-12 and sodium deoxycholate.

Solubilisation may also carried out by incubating membranes in a solubilisation buffer comprising Tris maleate, CaCl₂, NaCl, and CHAPS overnight at 4° C. The detergent suspension may then be centrifuged to recover proteins from the membrane preparation in the supernatant.

Mitochondria and inverted inner membrane vesicles may be prepared as described by Williams et al (5).

Lipoproteins—such as VLDL, LDL, HDL₂ and HDL₃—may be prepared by isolating them from the plasma of normolipidemic donors as previously described (6). ApoA-I may be isolated from HDL₃ by ion-exchange chromatography (7) and the purity may be assessed by SDS-PAGE and Western blot analysis (6). HDL₂ may be enriched in triacylglycerol as previously described (8).

Preferably, the sample is contacted with immobilised lipoprotein. The lipoprotein may be immobilised using various methods known in the art—such as traditional amine coupling chemistry (11).

Preferably, the sample is contacted with immobilised lipoprotein using Surface Plasmon Resonance. SPR is based upon electron charge density waves that occur at the surface of a metallic film when light is reflected at the film under certain conditions. The resonance is a result of energy and momentum being transformed from photons into surface plasmons, and is sensitive to the refractive index of the medium on the opposite side of the film from the reflected light. SPR was initially observed by Turbadar (1959) Proc. Phys. Soc. (London) 73; 40. Therefore, SPR may be used to monitor interactions occurring in a biospecific surface on a metal layer by measuring changes in the solute concentration at this surface as a result of the interactions. SPR is reviewed in Welford (1991) Opt. Quant. Elect. 23; 1 and Raether, H. (1977) Physics of Thin Films 9; 145.

Preferably, the sample may also be contacted with lipoprotein coupled to a support to affinity-purify the lipoprotein binding protein(s) by applying to a lipoprotein bead column.

If Surface Plasmon Resonance is used then the APROG microrecovery procedure (Biacore AB, Uppsala, Sweden) may be used to recover captured proteins. This procedure works by injecting a series of small liquid volumes separated by air bubbles over the sensor surface. Three liquid segments may be used: (1) Wash solution (20 μl) to rinse running buffer from the tubing and sensor surface; (2) Recovery plug (3-7 μl of recovery solution) to elute the bound analyte from the surface and (3) An additional segment of recovery solution (5 μl) to prevent contamination of the recovered sample with running buffer. This segment does not come into contact with the sensor surface. The command washes the flow cells with a user-defined solution and injects a plug of regeneration solution (sandwiched between air bubbles) onto the sensor surface. The liquid flow is stopped for a user-specified length of time while the recovery solution is in contact with the sensor surface. The flow direction is then reversed and the recovery solution containing eluted analyte is dispensed into a vial. The volume of recovery solution is sufficient to cover all four flow cells in multi-channel mode.

For the binding activity measurements of proteins eluted from apoA-I affinity chromatography, eluates may be diluted and injected in the first flow cell. As a control experiment, total solubilised porcine liver plasma membrane proteins may be diluted in running buffer and injected in the second flow cell.

If affinity-purification is used, washes with a suitable buffer—such as 10 mM triethylamine and 6M urea pH 11—may be used to elute bound proteins.

The eluates that are collected may be concentrated using various methods known to a person skilled in the art.

Various methods may be used by a person skilled in the art to determine if one or more lipoprotein bound proteins comprise at least one domain of ATP synthase.

By way of example, one or more lipoprotein bound proteins may be subjected to SDS/PAGE and the SDS/PAGE gel stained using various methods known in the art. Preferably, the SDS/PAGE gel is stained using silver staining or amidoblack staining. Stained bands may be cut out and digested, for example, with endoprotease lysine-C. The resulting peptides may be separated using various methods known in the art—such as by HPLC on a Cl 8 column with a 2-70% gradient of acetonitrile in 0.1% trifluoroacetic acid—and then sequenced.

Cell surface ADP and ATP measurements may also be performed. For example, cells are incubated with radiolabelled ATP for ADP generation assays, or with radiolabelled ADP for ATP generation assays. Depending on the experiment, IF₁ may be added in the reaction mixture. Supernatants may then be removed and analysed using various methods—such as by HPLC coupled to a radioactivity detector or by thin layer chromatography.

Other methods such as Western blotting (using an anti-ATP synthase antibody) and PCR (using ATP synthase specific primers) may also be used to determine if the lipoprotein bound proteins comprise at least one domain of ATP synthase.

The affinity of an unlabelled ligand for a receptor may be determined by measuring its ability to compete with a radioactive ligand for the receptor. Therefore, to confirm the association between HDL—such as free-apoA-I—and ATP synthase, competition experiments may be performed. In a competition experiment various concentrations of an unlabeled ligand are allowed to compete with a fixed concentration of a radiolabelled ligand for a receptor—such as an HDL receptor. As the concentration of unlabeled ligand increases, the amount of radioligand bound to the receptor decreases. The competitive inhibitor may be an agonist or an antagonist.

By way of example, the competition experiments may be performed as previously described (6). Briefly, cell monolayers or submitochondrial particles are incubated in a buffer—such as PBS—with a constant concentration of labeled ligand—such as HDL3 and free-apoA-I) and increasing concentrations of unlabeled competitors. Cells are washed, lysed and used for radioactivity measurement and protein determination. Submitochondrial particles are filtered washed as previously described (12). Filters are used for radioactivity measurements.

Optionally, the method may comprise the additional step of determining if at least one domain of ATP synthase is localised at the surface of a cell.

Various methods may be used to determine if at least one domain of ATP synthase is localised at the surface of a cell—such as immunofluorescence microscopy or fluorescence-assisted flow cytometry, as previously described.

Preferably, immunofluorescence microscopy or fluorescence-assisted flow cytometry are performed with an anti-ATP synthase monoclonal antibody. More preferably, the antibody is an anti-β-subunit ATP synthase monoclonal antibody.

Typically, experiments may be performed using a negative control—such as CHO cells.

Preferably, the lipoprotein bound proteins comprise one or more subunits of the F₁ domain of ATP synthase. More preferably, the lipoprotein bound proteins comprise the beta-subunit of the F₁ domain of ATP synthase.

Preferably, the beta-subunit of the F₁ domain of ATP synthase comprises SEQ ID No. 1 or a variant, derivative or homologue thereof.

Preferably, the beta-subunit of the F₁ domain of ATP synthase comprises a polypeptide encoded by SEQ ID No.2 or a variant, derivative or homologue thereof.

Assay Method

In a further aspect, the present invention provides an assay method for identifying one or more agents that modulate lipoprotein endocytosis.

Suitably, the assay method may be used to identify an agent—such as one or more agents—that is an antagonist of a lipoprotein receptor that decreases, reduces or diminishes the ability of a lipoprotein receptor ligand—such as free Apo A1—to bind to the lipoprotein receptor of the present invention. The assay method may also be used to identify an agent that is an antagonist of a lipoprotein receptor that decreases, reduces or diminishes lipoprotein endocytosis. The antagonists may be, for example, natural or modified substrates, ligands, receptors or enzymes or structural or functional mimetics thereof. For example, a cell—such as a hepatocyte—expressing the lipoprotein receptor of the present invention may be contacted with an agent. The ability of the agent to decrease lipoprotein endocytosis following addition of the agent is then measured.

By way of example, the antagonist of the lipoprotein receptor may be IF1, which is a natural inhibitor of the ATP hydrolysis of mitcohondirtal ATP synthase (7). As described herein, lipoprotein internalisation is decreased in the presence of IF1 protein.

Preferably, the assay method of the present invention is used to identify an agent that is an agonist of a lipoprotein receptor that potentiates, enhances or increases the ability of a lipoprotein receptor ligand—such as free Apo A1—to bind to the lipoprotein receptor of the present invention. The assay method may be used to identify an agent that is an agonist of a lipoprotein receptor that potentiates, enhances or increases lipoprotein endocytosis. The agonists may be, for example, natural or modified substrates, ligands, receptors or enzymes or structural or functional mimetics thereof. For example, a cell such as a hepatocyte—expressing the lipoprotein receptor of the present invention may be contacted with an agent. The ability of the agent to increase lipoprotein endocytosis following addition of the agent is then measured.

Preferably, the agent is an agonist of a lipoprotein receptor that potentiates, enhances or increases the ability of a lipoprotein receptor ligand—such as free Apo A1—to bind to the lipoprotein receptor of the present invention.

Preferably, the agonist may decrease, reduce or diminish the inhibitory activity of IF1 on the ATP hydrolysis of ATP synthase and so lipoprotein endocytosis is increased.

Fusion proteins, may be used for high-throughput screening assays to identify modulators of lipoprotein endocytosis (see D. Bennett et al., J Mol Recognition, 8: 52-58 (1995); and K. Johanson et al., J Biol Chem, 270(16): 9459-9471 (1995)).

Another technique for screening provides for high throughput screening (HTS) of agents having suitable binding affinity and is based upon the method described in detail in WO 84/03564.

For a general reference on screening, see the Handbook of Drug Screening, edited by Ramakrishna Seethala, Prabhavathi B. Fernandes. New York, N.Y., Marcel Dekker, 2001 (ISBN 0-8247-0562-9).

It is expected that the assay methods of the present invention will be suitable for both small and large-scale screening of agents as well as in quantitative assays.

The screening method may measure the binding of an agent to the lipoprotein receptor of the present invention by means of a label directly or indirectly associated with the agent. Alternatively, the screening method may involve competition with a labelled competitor.

A plurality of agents may be screened using the methods described below. In particular, these methods may be suited for identifying one or more agents that modulate lipoprotein endocytosis and for screening libraries of agents.

Where the candidate compounds are proteins e.g. antibodies or peptides, libraries of candidate compounds may be screened using phage display techniques. Phage display is a protocol of molecular screening, which utilises recombinant bacteriophage. The technology involves transforming bacteriophage with a gene that encodes the library of candidate compounds, such that each phage or phagemid expresses a particular candidate compound. The transformed bacteriophage (which preferably is tethered to a solid support) expresses the appropriate candidate compound and displays it on their phage coat. Specific candidate compounds which are capable of interacting with the lipoprotein receptor of the present invention are enriched by selection strategies based on affinity interaction. The successful candidate agents are then characterised. Phage display has advantages over standard affinity ligand screening technologies. The phage surface displays the candidate agent in a three dimensional configuration, more closely resembling its naturally occurring conformation. This allows for more specific and higher affinity binding for screening purposes.

Another method of screening a library of compounds utilises eukaryotic or prokaryotic host cells, which are stably transformed with recombinant DNA molecules expressing the library of compounds. Such cells, either in viable or fixed form, can be used for standard binding-partner assays. See also Parce et al. (1989) Science 246:243-247; and Owicki et al. (1990) Proc. Nat'l Acad. Sci. USA 87;4007-4011, which describe sensitive methods to detect cellular responses. Competitive assays are particularly useful, where the cells expressing the library of compounds are incubated with a labelled antibody, such as ¹²⁵I-antibody, and a test sample such as a candidate compound whose binding affinity to the binding composition is being measured. The bound and free labelled binding partners are then separated to assess the degree of binding. The amount of test sample bound is inversely proportional to the amount of labelled antibody bound.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Another technique for candidate compound screening involves an approach, which provides high throughput screening for new compounds having suitable binding affinity and is described in detail in WO 84/03564. First, large numbers of different small peptide agents are synthesised on a solid substrate, e.g., plastic pins or some other appropriate surface. Then all the pins are reacted with solubilised protein—such as solubilised membrane proteins comprising the lipoprotein receptor—and washed. The next step involves detecting bound protein. Detection may be accomplished using a monoclonal antibody. Compounds which interact specifically with the protein may thus be identified.

Rational design of candidate compounds likely to be able to interact with the lipoprotein receptor may be based upon structural studies of the molecular shapes of the protein and/or its in vivo binding partners. One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

One or more agents that affect the modulation of lipoprotein endocytosis—such as antagonists and agonists—may be identified by screening compounds. Such a method may comprise the steps of mixing a solution comprising an agent and the lipoprotein receptor of the present invention to form a mixture, and determining whether the ability of the agent to bind to the lipoprotein receptor of the present invention is altered.

Preferably, the assay method comprises the steps of identifying one or more agents that modulate ATP hydrolysis; and determining if the one or more agents modulate the activity of a lipoprotein receptor.

ATP hydrolysis may be measured using various methods known in the art. By way of example, cells may be incubated with 0.1 μCi [α-32P] ATP for ADP generation assays, or with 0.1 μCi 32Pi and ADP (100 nM final) for ATP generation assays. The agent may then be added in the reaction mixture. Supernatants may then be removed and analysed using various systems—such as 1) by HPLC coupled to a radioactivity detector on a Whatman Partisphere 5 SAX column (Whatman International Ltd., UK) as described previously (13); calibration may be done with radiolabelled nucleotides. 2) by thin layer chromatography in the solvent NaCl 2.4%/NH₄OH/H₂O/MeOH (12.5/15/27.5/50, v/v); radioactive spots may be counted by liquid scintillation.

Preferably, ATP is hydrolysed by at least one domain of ATP synthase.

Preferably, the lipoprotein receptor modulates lipoprotein endocytosis—such as HDL endocytosis.

Lipoprotein endocytosis may be determined using various methods in the art. By way of example, cells are washed and incubated with radiolabelled lipoprotein—such as ¹²⁵I triglyceride rich-HDL2—for various times. Depending on the experiment, free apolipoprotein A-I and other compounds—such as agents—are added to the incubation medium. At each incubation time, cells are washed and the release of radioactive ligands associated to the cell surface is measured. Briefly, the radioactivity internalised into the cells and the protein content is determined by washing the cells twice with ice-old PBS, lysing the cells with, for example, NaOH, and the NaOH digest may then used for radioactivity measurement and protein determination. Sub-mitochondrial particles are filtered on 0.22 μm filters (GVWP Millipore—France) and washed as previously described (12). Filters are used for radioactivity measurements. Non-specific internalisation may be analysed in the presence of an excess of 600 μg/ml of HDL3.

Preferably, the assay method is used to screen for agents that are useful in the treatment and/or prevention of diseases for example, cardiovascular disease, coronary heart disease, stroke, pancreatitis, atherosclerosis, gout, and/or type 2 diabetes.

The present invention also relates to an assay method to identify agents that modulate ATP hydrolysis, comprising the step of contacting ATPase with an agent in the presence of ATP and measuring the effect of the agent on ATP hydrolysis.

Preferably, the assay method is performed on a plasma membrane-bound ATPase.

Modulating Lipoprotein Endocytosis

The term “modulating lipoprotein endocytosis” may refer to preventing, suppressing, alleviating, restorating, elevating, increasing or otherwise affecting lipoprotein endocytosis in a cell.

Preferably, the term refers to restorating, elevating or increasing lipoprotein endocytosis in a cell.

Thus, in a further aspect, the present invention relates to assay methods, processes, and agents that modulate lipoprotein endocytosis or affect the modulation of lipoprotein endocytosis. Preferably, the assay methods, processes, and agents restore, elevate or increase lipoprotein endocytosis.

Agents that affect the modulation of lipoprotein endocytosis may affect the activity and/or expression of the lipoprotein receptor of the present invention or a ligand thereof. By way of example, if the agent is a lipoprotein receptor ligand—such as a HDL receptor ligand—then this ligand may bind to the lipoprotein receptor of the present invention and increase its activity such that an increased level of cholesterol is extracted from peripheral cells by HDL and delivered to the liver for its elimination. By way of a further example, the agent may bind to the nucleotide sequence encoding the lipoprotein receptor described herein, or control regions associated with the nucleotide coding sequence, or its corresponding RNA transcript to modify (eg. increase) the rate of transcription or translation.

Other methods may also be employed, so long as their affect is to modulate lipoprotein endocytosis. Such methods may include modulation of expression, activity or degradation of any element, which ultimately results in lipoprotein endocytosis. The expression of the lipoprotein receptor of the present invention may be modulated, using, for example, antisense oligonucleotides to an mRNA encoding the lipoprotein receptor of the present invention. The expression of the lipoprotein receptor may also be modulated by modulating the transcription of such an mRNA, or by modulating mRNA processing etc. Translation of the lipoprotein receptor protein from lipoprotein receptor mRNA may also be regulated as a means of modulating the expression of this protein. Such modulation may make use of methods known in the art, for example, by use of agents that are inhibitors of transcription or translation.

Such agents may even modulate the activity of a further entity.

If an agent modulates lipoprotein endocytosis by decreasing lipoprotein endocytosis then it may be known as an antagonist.

If an agent modulates lipoprotein endocytosis by increasing lipoprotein endocytosis then it may be known as an agonist. Preferably, the agents that modulate endocytosis according to the present invention are agonists.

The agents that modulate lipoprotein endocytosis may be used for manufacturing pharmaceutical compositions, which may be used in medicine, in particular for the treatment and/or prevention of diseases, for example, cardiovascular disease, coronary heart disease, stroke, pancreatitis, atherosclerosis, gout, and/or type 2 diabetes.

In a further aspect, the present invention relates to a process comprising the steps of: performing the assay method of the present invention; identifying an agent capable of modulating lipoprotein endocytosis; and preparing a quantity of that agent.

Agent

The agent according to the present invention may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may even be a polynucleotide molecule—which may be a sense or an anti-sense molecule, or an antibody, for example, a polyclonal antibody, a monoclonal antibody or a monoclonal humanised antibody.

Various strategies have been developed to produce monoclonal antibodies with human character, which bypasses the need for an antibody-producing human cell line. For example, useful mouse monoclonal antibodies have been “humanised” by linking rodent variable regions and human constant regions (Winter, G. and Milstein, C. (1991) Nature 349, 293-299). This reduces the human anti-mouse immunogenicity of the antibody but residual immunogenicity is retained by virtue of the foreign V-region framework. Moreover, the antigen-binding specificity is essentially that of the murine donor. CDR-grafting and framework manipulation (EP 0239400) has improved and refined antibody manipulation to the point where it is possible to produce humanised murine antibodies which are acceptable for therapeutic use in humans. Humanised antibodies may be obtained using other methods well known in the art (for example as described in US-A-239400).

The agent may even be improved analogues of agents that modulate lipoprotein endocytosis.

The agents may be attached to an entity (e.g. an organic molecule) by a linker which may be a hydrolysable bifunctional linker.

The entity may be designed or obtained from a library of compounds, which may comprise peptides, as well as other compounds, such as small organic molecules.

By way of example, the entity may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a peptide cleaved from a whole protein, or a peptides synthesised synthetically (such as, by way of example, either using a peptide synthesizer or by recombinant techniques or combinations thereof, a recombinant agent, an antibody, a natural or a non-natural agent, a fusion protein or equivalent thereof and mutants, derivatives or combinations thereof.

Typically, the entity will be an organic compound. For some instances, the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the entity comprises at least one cyclic group. The cyclic group may be a polycyclic group, such as a non-fused polycyclic group. For some applications, the entity comprises at least the one of said cyclic groups linked to another hydrocarbyl group.

The entity may contain halo groups—such as fluoro, chloro, bromo or iodo groups. The entity may contain one or more of alkyl, alkoxy, alkenyl, alkylene and alkenylene groups—which may be unbranched- or branched-chain.

Prodrug

It will be appreciated by those skilled in the art that the entity may be derived from a prodrug. Examples of prodrugs include certain protected group(s) which may not possess pharmacological activity as such, but may, in certain instances, be administered (such as orally or parenterally) and thereafter metabolised in the body to form an entity that is pharmacologically active.

Suitable pro-drugs may include, but are not limited to, Doxorubicin, Mitomycin, Phenol Mustard, Methotraxate, Antifolates, Chloramphenicol, Camptothecin, 5-Fluorouracil, Cyanide, Quinine, Dipyridamole and Paclitaxel. Agents (e.g. an antibody or a fragment thereof) that bind the lipoprotein receptor identified using the methods of the present invention may be chemically linked to an enzyme of interest. Alternatively, the conjugate can be a fusion protein produced by recombinant DNA techniques with the antibody variable region genes and the gene encoding the enzyme. Preferably, the prodrug should be non-toxic, resistant to the action of endogenous enzymes, and be converted into active drug only by the targeted enzyme. The selective activation of anticancer prodrugs by mAb-enzyme conjugates is reviewed in Senetr & Springer (2001) Advanced Drug Delivery Reviews 53, 247-264.

It will be further appreciated that certain moieties known as “pro-moieties”, for example as described in “Design of Prodrugs” by H. Bundgaard, Elsevier, 1985, may be placed on appropriate functionalities of the agents. Such prodrugs are also included within the scope of the invention.

The agent may be in the form of a pharmaceutically acceptable salt—such as an acid addition salt or a base salt—or a solvate thereof, including a hydrate thereof. For a review on suitable salts see Berge et al, J. Pharm. Sci., 1977, 66, 1-19. The agent of the present invention may be capable of displaying other therapeutic properties.

The agent may be used in combination with one or more other pharmaceutically active agents.

If combinations of active agents are administered, then the combinations of active agents may be administered simultaneously, separately or sequentially.

Stereo and Geometric Isomers

The entity may exist as stereoisomers and/or geometric isomers—e.g. the entity may possess one or more asymmetric and/or geometric centres and so may exist in two or more stereoisomeric and/or geometric forms. The present invention contemplates the use of all the individual stereoisomers and geometric isomers of those entities, and mixtures thereof.

Pharmaceutical Salt

The agents of the present invention may be administered in the form of a pharmaceutically acceptable salt.

Pharmaceutically-acceptable salts are well known to those skilled in the art, and for example, include those mentioned by Berge et al, in J. Pharm. Sci., 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

A pharmaceutically acceptable salt of an agent may be readily prepared by mixing together solutions of the agent and the desired acid or base, as appropriate. The salt may precipitate from solution and be collected by filtration or may be recovered by evaporation of the solvent.

The agent of the present invention may exist in polymorphic form.

The agent of the present invention may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The agent may also include all suitable isotopic variations of the agent or a pharmaceutically acceptable salt thereof. An isotopic variation of an agent or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, 15N, ¹⁷O, ¹⁸O, ³¹P, ³²P, 35S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent and pharmaceutically acceptable salts thereof of this invention can generally be prepared by conventional procedures using appropriate isotopic variations of suitable reagents.

Pharmaceutically Active Salt

The agent may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

Chemical Synthesis Methods

The agent may be prepared by chemical synthesis techniques.

It will be apparent to those skilled in the art that sensitive functional groups may need to be protected and deprotected during synthesis of a compound of the invention. This may be achieved by conventional techniques, for example, as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be racemised, for example, if a base is used in a reaction with a substrate having an having an optical centre comprising a base-sensitive group. This is possible during e.g. a guanylation step. It should be possible to circumvent potential problems such as this by choice of reaction sequence, conditions, reagents, protection/deprotection regimes, etc. as is well-known in the art.

The compounds and salts may be separated and purified by conventional methods.

Separation of diastereomers may be achieved by conventional techniques, e.g. by fractional crystallisation, chromatography or H.P.L.C. of a stereoisomeric mixture of a compound of formula (I) or a suitable salt or derivative thereof. An individual enantiomer of a compound of formula (I) may also be prepared from a corresponding optically pure intermediate or by resolution, such as by H.P.L.C. of the corresponding racemate using a suitable chiral support or by fractional crystallisation of the diastereomeric salts formed by reaction of the corresponding racemate with a suitably optically active acid or base.

The agent or variants, homologues, derivatives, fragments or mimetics thereof may be produced using chemical methods to synthesise the agent in whole or in part. For example, if the agent comprises a peptide, then the peptide can be synthesised by solid phase techniques, cleaved from the resin, and purified by preparative high performance liquid chromatography (e.g., Creighton (1983) Proteins Structures And Molecular Principles, W H Freeman and Co, New York N.Y.). The composition of the synthetic peptides may be confirmed by amino acid analysis or sequencing (e.g., the Edman degradation procedure; Creighton, supra).

Synthesis of peptide inhibitor agents (or variants, homologues, derivatives, fragments or mimetics thereof) can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the agent, may be altered during direct synthesis and/or combined using chemical methods with a sequence from other subunits, or any part thereof, to produce a variant agent.

Chemical Derivative

The term “derivative” or “derivatised” as used herein includes chemical modification of an agent. Illustrative of such chemical modifications would be replacement of hydrogen by a halo group, an alkyl group, an acyl group or an amino group.

Chemical Modification

The agent may be a modified agent—such as, but not limited to, a chemically modified agent.

The chemical modification of an agent may either enhance or reduce hydrogen bonding interaction, charge interaction, hydrophobic interaction, Van Der Waals interaction or dipole interaction.

In one aspect, the agent may act as a model (for example, a template) for the development of other compounds.

Pharmaceutical Compositions

Pharmaceutical compositions of the present invention may comprise a therapeutically effective amount of the agent.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

If the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions may be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or the pharmaceutical compositions can be injected parenterally, for example, intravenously, intramuscularly or subcutaneously. For parenteral administration, the compositions may be best used in the form of a sterile aqueous solution which may contain other substances, for example, enough salts or monosaccharides to make the solution isotonic with blood. For buccal or sublingual administration the compositions may be administered in the form of tablets or lozenges which can be formulated in a conventional manner.

The agents may be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A98/55148.

If the agent is a protein, then said protein may be prepared in situ in the subject being treated. In this respect, nucleotide sequences encoding said protein may be delivered by use of non-viral techniques (e.g. by use of liposomes) and/or viral techniques (e.g. by use of retroviral vectors) such that the said protein is expressed from said nucleotide sequence.

The pharmaceutical composition comprising the receptor of the present invention or a variant, homologue, fragment or derivatives thereof may also be used in combination with conventional treatments of cholesterolameia—such as lifestyle modification, including cessation of cigarette smoking, weight reduction, regular phsyical exercise and possibly a moderate intake of alcohol (Ginsberg (2000) Am. J. Cardiol. 86 41L-45L). A statin may be employed to lower LDL-cholesterol to, for example, below 2.6 nmol/l; if HDL still remains below 0.9 nmol/l with or without elevation of triglycerides, then a fibrate may be used as adjunctive therapy. Antihypertensive and antidiabetic agents may also be used.

In a further aspect, the present invention relates to a process comprising the steps of: (i) performing the assay according to the present invention; (ii) identifying an agent capable of modulating lipoprotein endocytosis; (iii) preparing a quantity of that agent; and (iv) preparing a pharmaceutical composition comprising that agent.

In still a further aspect, the present invention relates to a process comprising the steps of: (i) performing the assay according to the present invention; (ii) identifying an agent capable of modulating lipoprotein endocytosis; (iii) modifying said agent; and (iv) preparing a pharmaceutical composition comprising said modified agent.

Administration

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The components may be administered alone but will generally be administered as a pharmaceutical composition—e.g. when the components are is in admixture with a suitable pharmaceutical excipient, diluent or carrier selected with regard to the intended route of administration and standard pharmaceutical practice.

For example, the components can be administered in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) may include, but are not limited to, one or more of oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

By way of example only, the components may be administered when triglyceride levels are below 2.2 mmol/l—such as 1.1 mmol/l, and HDL cholesterol is greater than 1 mmol/l—such as above 1.2 mmol/l.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Formulation

The component(s) may be formulated into a pharmaceutical composition, such as by mixing with one or more of a suitable carrier, diluent or excipient, by using techniques that are known in the art.

Diseases

Aspects of the present invention may be used for the treatment or prevention of diseases associated with modulated—such as increased—levels of cholesterol. For example, the disease may be cardiovascular diseases, coronary heart disease, stroke, pancreatitis, atherosclerosis, gout, and/or type 2 diabetes.

Preferably, the disease is atherosclerosis or coronary heart disease.

Cholesterol levels may be diagnosed using a total serum cholesterol test in which a small amount of blood (eg. 5 millilitres) is withdrawn.

Nucleotide Sequence

The present invention involves the use of nucleotide sequences, which may be available in databases. These nucleotide sequences may be used to express amino acid sequences.

Preferably, the lipoprotein receptor is encoded by SEQ ID No.2 or a variant, derivative homologue or fragment thereof.

The nucleotide sequence may be DNA or RNA of genomic, synthetic or recombinant origin e.g. cDNA. The nucleotide sequence may be double-stranded or single-stranded whether representing the sense or antisense strand or combinations thereof.

The nucleotide sequence may be prepared by use of recombinant DNA techniques (e.g. recombinant DNA).

The nucleotide sequence may be the same as the naturally occurring form, or may be derived therefrom.

The nucleotide sequence may be a nucleotide sequence of interest i.e. a nucleotide sequence representing the coding sequence of the protein product, incorporating its own termination codon, but minus the native signal sequence.

Amino Acid

Aspects of the present invention concern the use of amino acid sequences, which may be available in databases. These amino acid sequences may comprise the agent of the present invention. In another embodiment, the amino acid sequences may be used as a target to identify suitable agents for use in the composition of the present invention. In another embodiment, the amino acid sequences may be used as a target to verify that an agent may be used as an agent according to the present invention.

Preferably, the lipoprotein receptor comprises SEQ ID No.1 or a variant, derivative homologue or fragment thereof.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

The amino acid sequence may be isolated from a suitable source, or it may be made synthetically or it may be prepared by use of recombinant DNA techniques.

Host Cells

As used herein, the term “host cell” refers to any cell that comprises nucleotide sequences that are of use in the present invention.

Host cells may be transformed or transfected with a nucleotide sequence contained in a vector e.g. a cloning vector. Preferably said nucleotide sequence is carried in a vector for the replication and/or expression of the nucleotide sequence. The cells will be chosen to be compatible with the said vector and may, for example, be prokaryotic (for example bacterial), fungal, yeast or plant cells.

The gram-negative bacterium E. coli is widely used as a host for cloning nucleotide sequences. This organism is also widely used for heterologous nucleotide sequence expression. However, large amounts of heterologous protein tend to accumulate inside the cell. Subsequent purification of the desired protein from the bulk of E. coli intracellular proteins can sometimes be difficult.

In contrast to E. coli, bacteria from the genus Bacillus are very suitable as heterologous hosts because of their capability to secrete proteins into the culture medium. Other bacteria suitable as hosts are those from the genera Streptomyces and Pseudomonas.

Depending on the nature of the polynucleotide and/or the desirability for further processing of the expressed protein, eukaryotic hosts including yeasts or other fungi may be preferred. In general, yeast cells are preferred over fungal cells because yeast cells are easier to manipulate. However, some proteins are either poorly secreted from the yeast cell, or in some cases are not processed properly (e.g. hyperglycosylation in yeast). In these instances, a different fungal host organism should be selected.

Examples of expression hosts are fungi—such as Aspergillus species (such as those described in EP-A-0184438 and EP-A-0284603) and Trichoderma species; bacteria—such as Bacillus species (such as those described in EP-A-0134048 and EP-A-0253455), Streptomyces species and Pseudomonas species; and yeasts—such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus orvzae, Trichoderma reesei, Bacillus subtilis, Bacillus licheniformis, Bacillus amyloliquefaciens, Kluyveromyces lactis and Saccharomyces cerevisiae.

The use of host cells—such as yeast, fungal and plant host cells—may provide for posttranslational modifications (e.g. myristoylation, glycosylation, truncation, lapidation and tyrosine, serine or threonine phosphorylation) as may be needed to confer optimal biological activity on recombinant expression products of the present invention.

Aspects of the present invention also relate to host cells comprising the expression vector of the present invention. The expression vector may comprise a nucleotide sequence for replication and expression of the sequence. The cells will be chosen to be compatible with the vector and may, for example, be prokaryotic (for example bacterial), fungal, yeast or plant cells.

Preferably, the host cells are mammalian cells—such as CHO cells.

Transfection

Introduction of a vector into a host cell can be effected by various methods. For example, calcium phosphate transfection, DEAE-dextran mediated transfection, cationic lipid-mediated transfection, electroporation, transduction or infection may be used. Such methods are described in many standard laboratory manuals—such as Sambrook et al., Molecular Cloning: A Laboratory Manual, 2d ed. (1989) Cold Spring Harbor Laboratory Press, Cold Spring Harbor, N.Y.

Host cells containing the expression vector can be selected by using, for example, G418 for cells transfected with an expression vector carrying a neomycin resistance selectable marker.

Transformation

Teachings on the transformation of cells are well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc.

If a prokaryotic host is used then the nucleotide sequence may need to be suitably modified before transformation—such as by removal of introns.

A host cell may be transformed with a nucleotide sequence. Host cells transformed with the nucleotide sequence may be cultured under conditions suitable for the replication or expression of the nucleotide sequence.

Constructs

Nucleotide sequences may be present in a construct.

The term “construct”—which is synonymous with terms such as “conjugate”, “cassette” and “hybrid”—includes a nucleotide sequence directly or indirectly attached to a promoter. An example of an indirect attachment is the provision of a suitable spacer group such as an intron sequence including the Sh1-intron or the ADH intron, intermediate to the promoter and the nucleotide sequence. The same is true for the term “fused” which includes direct or indirect attachment. In some cases, the terms do not cover the natural combination of the nucleotide sequence coding for the protein ordinarily associated with the wild type nucleotide sequence promoter and when they are both in their natural environment.

The construct may even contain or express a marker, which allows for the selection of the nucleotide sequence construct in, for example, a bacterium, preferably of the genus Bacillus, such as Bacillus subtilis, or plants into which it has been transferred. Various markers exist which may be used, for example those encoding mannose-6-phosphate isomerase (especially for plants) or those markers that provide for antibiotic resistance—e.g. resistance to G418, hygromycin, bleomycin, kanamycin and gentamycin.

Vectors

Nucleotide sequences may be present in a vector.

The term “vector” includes expression vectors and transformation vectors and shuttle vectors.

The term “transformation vector” means a construct capable of being transferred from one entity to another entity—which may be of the species or may be of a different species. If the construct is capable of being transferred from one species to another e.g. from an E. coli plasmid to a bacterium, such as of the genus Bacillus, then the transformation vector is sometimes called a “shuttle vector”. It may even be a construct capable of being transferred from an E. coli plasmid to an Agrobacterium to a plant.

The vectors may be transformed into a suitable host cell as described below to provide for expression of a polypeptide.

The vectors may be for example, plasmid, virus or phage vectors provided with an origin of replication, optionally a promoter for the expression of the said polynucleotide and optionally a regulator of the promoter.

The vectors may contain one or more selectable marker nucleotide sequences. The most suitable selection systems for industrial micro-organisms are those formed by the group of selection markers which do not require a mutation in the host organism. Examples of fungal selection markers are the nucleotide sequences for acetamidase (amdS), ATP synthetase, subunit 9 (oliC), orotidine-5′-phosphate-decarboxylase (pvrA), phleomycin and benomyl resistance (benA). Examples of non-fungal selection markers are the bacterial G418 resistance nucleotide sequence (this may also be used in yeast, but not in filamentous fungi), the ampicillin resistance nucleotide sequence (E. coli), the neomycin resistance nucleotide sequence (Bacillus) and the E. coli uidA nucleotide sequence, coding for β-glucuronidase (GUS).

Vectors may be used in vitro, for example for the production of RNA or used to transfect or transform a host cell.

Thus, polynucleotides may be incorporated into a recombinant vector (typically a replicable vector), for example, a cloning or expression vector. The vector may be used to replicate the nucleic acid in a compatible host cell.

Variants/Homologues/Derivatives

The present invention encompasses the use of variants, homologues, derivatives and fragments thereof.

The term “variant” is used to mean a naturally occurring polypeptide or nucleotide sequences which differs from a wild-type sequence.

The term “fragment” indicates that a polypeptide or nucleotide sequence comprises a fraction of a wild-type sequence. It may comprise one or more large contiguous sections of sequence or a plurality of small sections. The sequence may also comprise other elements of sequence, for example, it may be a fusion protein with another protein. Preferably the sequence comprises at least 50%, more preferably at least 65%, more preferably at least 80%, most preferably at least 90% of the wild-type sequence.

The term “homologue” means an entity having a certain homology with the subject amino acid sequences and the subject nucleotide sequences. Here, the term “homology” can be equated with “identity”.

In the present context, a homologous sequence is taken to include an amino acid sequence, which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence, which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons may be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix—such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues, which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example, according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution—such as basic for basic, acidic for acidic, polar for polar etc. Non-homologous substitution may also occur i.e. from one class of residue to another or alternatively involving the inclusion of unnatural amino acids—such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids—such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#*}, L-norleucine*, L-norvaline*, p-nitro-L-phenylalanine*, L-hydroxyproline*, L-thioproline*, methyl derivatives of phenylalanine (Phe)—such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid* and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups—such as methyl, ethyl or propyl groups—in addition to amino acid spacers—such as glycine or β-alanine residues. A further form of variation involves the presence of one or more amino acid residues in peptoid form will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example, Simon RJ et al., PNAS (1992) 89(20), 9367-9371 and Horwell DC, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences may be modified by any method available in the art. Such modifications may be carried out to enhance the in vivo activity or life span of nucleotide sequences useful in the present invention.

The present invention may also involve the use of nucleotide sequences that are complementary to the nucleotide sequences or any derivative, fragment or derivative thereof. If the sequence is complementary to a fragment thereof then that sequence can be used as a probe to identify similar coding sequences in other organisms etc.

Gene Therapy

The present invention encompasses gene therapy whereby nucleotide sequences coding for the lipoprotein receptor of the present invention are regulated in vivo. For example, regulation of expression may be accomplished by administering compounds that bind to the nucleotide coding sequence, or control regions associated with the nucleotide coding sequence for the lipoprotein receptor, or its corresponding RNA transcript to modify the rate of transcription or translation.

By way of example, a nucleotide sequence encoding a lipoprotein receptor according to the present invention, may be under the control of an expression regulatory element—such as a promoter or a promoter and enhancer. The enhancer and/or promoter may even be active in particular tissues, such that the nucleotide sequence coding for the receptor of the present invention is preferentially expressed. The enhancer element or other elements conferring regulated expression may be present in multiple copies. Likewise, or in addition, the enhancer and/or promoter may be preferentially active in one or more specific cell types—such as hepatocytes.

The level of expression of the nucleotide sequence coding for the lipoprotein receptor of the present invention, may be modulated by manipulating the promoter region. For example, different domains within a promoter region may possess different gene regulatory activities. The roles of these different regions are typically assessed using vector constructs having different variants of the promoter with specific regions deleted (that is, deletion analysis).

General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### DESCRIPTION OF THE FIGURES

FIG. 1

Affinity purification of free-apoA-I receptor.

(A) Affinity purification of p50. The total solubilised porcine liver plasma membrane proteins (lane 1) are either subjected to apoA-I affinity chromatography (lane 2) or injected on the apoA-I sensor chip BI and subjected to micro-recovery (lane 3). Proteins are eluted and samples (lane 1 and 2, 5 μg of protein; lane 3, 0.1 μg of proteins) are separated on SDS/PAGE, followed by silver staining.

(B) Biacore sensograms of the interaction of total solubilised porcine liver plasma membrane proteins directly injected (1, dashed line), or recovered by apoA-I affinity chromatography (2, solid line), with apoA-I sensor chip BI. 1.5 μg proteins are injected in HBS (running buffer) at a flow rate of 20 μl/min. Dissociation is observed in running buffer at the same flow rate. Curves represent the resonance unit as a function of time.

FIG. 2

Immunofluorescence localisation of the β-subunit of ATP synthase and apoA-I on the surface of hepatocytes.

Co localization of the b-subunit of ATP synthase (green, panel A) and the apoA-I (red, panel B) on intact IHH was evidenced. Panel C is the merged image of panels A and B. Localization of the a-chain of ATP synthase was performed by immunofluorescence using anti a-chain IgG on intact IHH (panel D). Anti subunit I of cytochrome oxidase (COX), another typical mitochondria protein, was used as control on intact (panel E) or permeabilized (panel H) IHH. b-subunit of ATP synthase was undetectable on intact CHO cells (panel F). Panels G (IHH) and I (CHO cells) were control experiments, performed with isotypic purified mouse IgG (IgG2a).

FIG. 3

Detection of the cell surface β-subunit of ATP synthase by flow cytometry HepG2 and CHO cells are analysed by fluorescence-assisted flow cytometry.

HepG2 and CHO cells were analyzed by fluorescence-assisted flow cytometry. Plots are shown for HepG2 (panel A) and CHO (panel C) where solid lines represent cells incubated with an anti b-subunit of ATP synthase mouse monoclonal IgG2a in absence (e) or presence (d) of 100 nM apoA-I, 250 μg/ml of FI-ATPase (c) or incubated with an isotypic control mouse IgG2a (a) and 20 anti cytochrome oxidase subunit I mouse monoclonal IgG2a (b, dashed lines). In panel B and D, the histograms are expressed as the mean relative fluorescence of the curves a to e on HepG2 (B) or CHO cells (D).

FIG. 4

Competitive inhibition of the binding of ¹²⁵1-labeled apoA-I to HepG2 cells and of ¹²⁵I-labeled HDL3 to submitochondrial particles.

HepG2 cells (panel A) and submitochondrial particles (panel B) are incubated for 2 h at 4° C. in the presence of 1 μg/ml ¹²⁵I-labeled apoA-1 and 5 μg/ml ¹²⁵I-labeled HDL3 respectively, in the presence of increasing concentrations of either unlabeled apoA-I (◯, panel A), unlabeled HDL3 (●, panel B), anti β-subunit of ATP synthase mouse monoclonal IgG2a (□) or purified isotype mouse IgG2a (♦). The 100% specific binding corresponds to 25,5 ng apoA-I bound per mg of cell proteins (panel A) and 2050 ng HDL3 protein bound per mg of mitochondrial proteins (panel B). Fab fragments from anti-β-subunit of ATP synthase are also used as competitors and results are identical to anti β-subunit IgG2a (data not shown). The results are representative of three independent series of experiments.

FIG. 5

H.P.L.C profiles of the nucleotides generated at the HepG2 cell surface.

³²Pᵢ and ADP (panels A and B) or [α-32P] ATP (panels C, D, E and F) are added to the HepG2 cell medium as described in Experimental Procedures, and the cells are incubated for 10 min at 37° C. with 10 μg/ml apoA-I (panels B and D), 100 nM IF₁ (panel E), or both IF₁ 17 and apoA-I at the same concentrations as above (panel F). Control experiments are performed with neither apo AI nor IF₁ added into the cell medium (panel A and C). Supernatants are recovered and analysed by HPLCs.

FIG. 6

Effect of different nucleotides on TG-HDL2 internalisation by hepatocytes.

Panel 1, 2 and 3. TG-HDL2 and LDL internalization by HepG2 cells. Cells are incubated for 10 min at 37° C. with 75 μg/ml of ¹²⁵I-TG-HDL2 (panels 1 and 3), TG-HDL2 labelled with ³H-cholesteryl ester (panel 1 “chol”) or ¹²⁵1-LDL (panel 2). Alternatively, 10 μg/ml free-apoA-I (A, G), 100 nM ADP (B, H), both free-apoA-I and ADP at the same concentrations as above (C), 100 nM ATP (D), 0.2 U/ml apyrase (E), 100 nM EGF (F), or increasing concentrations of 2MeS-ADP (▪) and ATPγS (□) (panel 3) are added to the incubation medium. Results are expressed as the percentage of stimulation related to the value of internalisation obtained without addition of any compound (corresponding to a value of 400 ng TG-HDL2/mg of cell protein). The same experiments are performed on IHH cells with similar results (data not shown).

Panel 4. Internalisation of EGF receptor by HepG2 cells. Cells are pre-incubated for 10 min in serum-free medium with (J) or without (K) 20 mM EGF, or with 10 μg/ml free-apoA-I (L), 100 nM ADP (M). The amount of EGF receptor is measured by Flow Cytometry using anti-EGF receptor antibody. As a control experiment for the efficacy of EGF receptor antibody, isotypic IgG are used (I). Results are expressed as the mean relative cell fluorescence and receptor internalisation is related to lower fluorescence.

FIG. 7

Effects of IF₁ on TG-HDL2 internalisation by hepatocytes.

HepG2 cells are incubated for 10 min at 37° C. in DMEM pH 6.6 with 75 μg/ml of ¹²⁵I-TGHDL 2 and in the presence of increasing concentrations of IF, without (panel A) or with (panel B) free-apoA-I (10 μg/ml). Results are expressed as the percentage of stimulation as referred to the value of internalisation obtained without addition of any compound (corresponding to a value of 400 ng TG-HDL2/mg of cell protein).

The invention will now be further described by way of Examples, which are meant to serve to assist one of ordinary skill in the art in carrying out the invention and are not intended in any way to limit the scope of the invention.

### EXAMPLES

Materials and Methods

Cells.

HepG2 cells and Chinese hamster ovary (CHO) cells are obtained from American Type Culture Collection (Rockville, Md., USA). Immortalised Human Hepatocytes (IHH) are obtained from Dr. Moshage (1). Primary cultures of adult human hepatocytes are provided by Dr. Maurel (2). HepG2 and IHH are cultured in Dulbecco's modified Eagles medium (DMEM) supplemented with penicillin/streptomycin and 10% fetal calf serum. CHO cells are maintained in Ham's F-12 medium supplemented with penicillin/streptomycin and 5% fetal calf serum.

Preparation of Porcine Liver Plasma Membranes and Submitochondrial Particle.

Plasma membranes are prepared by the aqueous two-phases partition procedure and according to previous reports, the dominant orientation is right-side-out (cytoplasmic side in) (3). Measurement of specific enzymatic markers confirm that the starting material is pure hepatocyte plasma membranes (4). Solubilisation is carried out by incubating membranes at a concentration of 1.5 mg of protein/ml in 125 mM Tris maleate, 1 mM CaCl2, 150 mM NaCl, 8 mM CHAPS, pH 7.4 (solubilisation buffer) overnight at 4° C. The detergent suspension is then centrifuged at 100,000×g for 1 h at 4° C. under these conditions, 50-60% of proteins from the membrane preparation are recovered in the supernatant. Mitochondria and inverted inner membrane vesicles are prepared as described by Williams et al (5).

Lipoprotein and Apolipoprotein Preparations.

VLDL, LDL, HDL2 and HDL3 are isolated from the plasma of normolipidemic donors as previously described (6). ApoA-I is isolated from HDL3 by ion-exchange chromatography (7) and the purity is assessed by SDS-PAGE and Western blot analysis (6). HDL2 is enriched in triacylglycerol as previously described (8). 3H-cholesteryl ester (CE) labelling of triglyceride rich-HDL2 (average 35000 dpm/μg CE) is realised as previously described (9) using a non-degradable 3H-cholesteryl-oleoyl ether. ¹²⁵I-labeling of free-apoA-I and lipoproteins is performed by the N-bromosuccinimide method (10). Specific radioactivities ranged from 3000 to 5000 cpm/ng of protein for lipoproteins, and from 10 000 to 20 000 cpm/ng of protein for apoA-I. More than 97% of the radioactivity is associated with proteins.

Surface Plasmon Resonance

Surface Plasmon Resonance measurements and recovery is performed at 20° C. using a BIACORE 3000 (Biacore AB, Uppsala, Sweden), equipped with a research-grade B1 sensor chip. ApoA-I (50 fmol/mm2) is immobilised on three flow cells using traditional aminecoupling chemistry (11). The fourth flow cell (control) is inactivated without immobilised apoA-I. Solubilised porcine liver plasma membrane proteins, prepared as above, are diluted 8 times in running buffer (10 mM HEPES, 150 mM NaCl, 3 mM EDTA, 0,005% Polysorbate, pH 7.4), then 100 μg of proteins are injected at a flow rate of 20 μl/min. APROG microrecovery procedure (Biacore AB, Uppsala, Sweden) is performed to recover captured proteins in 7 μl of elution buffer (10 mM Triethylamine, 6M Urea, pH 11). The eluates are subjected to SDS/PAGE, followed by silver staining. For the binding activity measurement of porcine liver plasma membrane proteins eluted from apoA-I affinity chromatography, eluates are diluted 3 times in running buffer and 1.5 μg of proteins is injected in the first flow cell. As a control experiment, 1.5 μg of total solubilised porcine liver plasma membrane proteins diluted 8 times in running buffer is injected in the second flow cell.

ApoA-I Affinity Chromatography and Peptide Sequence Analysis.

Apolipoprotein AI, coupled to Affi-gel 15 support (Bio-Rad Laboratories), is used to affinity-purify the apoA-I-binding protein(s). Solubilized porcine liver plasma membrane extracts is diluted to a final concentration of 1 mM CHAPS and then applied on the apoA-I bead column for 1 h at 4° C. Following five washes with 10 ml of 0.1M sodium acetate buffer pH 6.5, bound proteins are eluted in 10 mM Triethylamine, 6M Urea, pH 11. The eluates are concentred in amicon Ultrafree-MC 10,000 NMWL (Millipore) and analyzed by SDS/PAGE, followed by silver or amidoblack staining. An amidoblack-stained band of 50 kDa is cut out and digested with endoprotease lysine-C. The resulting peptides are separated by HPLC on a C18 column with a 2-70% gradient of acetonitrile in 0.1% trifluoroacetic acid and then sequenced (Institut Pasteur, Paris, France).

Competition Assays.

The competition experiments are performed at 4° C. for 2 h as previously described (6). Briefly, cell monolayers (300 000 cells/well) or sub-mitochondrial particles (50 μg of proteins) are incubated in PBS for 2 h at 4° C. with a constant concentration of labelled ligand (5 μg/ml for HDL3 and 1 μg/ml for free-apoA-I) and in the presence of increasing concentrations of unlabeled competitors. Cells are washed twice with ice-cold PBS (maximum washing time is 15 s), lysed with 500 μl of 0.1 N NaOH, and the NaOH digest is used for radioactivity measurement and protein determination. Sub-mitochondrial particles are filtered on 0.22 μm filters (GVWP Millipore—France) and washed three times with 1% BSA in PBS as previously described (12). Filters are used for radioactivity measurements. Non specific binding represented 35-40% of total binding. Data are expressed as the percentage of the specific binding measured in the absence of competitor versus the log of competitor concentration (in nM).

Internalisation Assays.

Cells are washed three times and pre-incubated for 30 min at 37° C. in serum free DMEM. ¹²⁵I triglyceride rich-HDL2 (75 μg/ml) are added on the cells and incubated at 37° C. for various time. Depending on the experiment, free apolipoprotein A-I (10 μg/ml) or other compounds, are added to the incubation medium. At each incubation time, cells are washed with cold DMEM and the release of radioactive ligands associated to the cell surface is performed at 4° C., for 90 min in DMEM. The radioactivity internalised into the cells and the protein content is determined as in the competition assays. Non-specific internalisation is analysed in the presence of an excess of 600 μg/ml of HDL3. Results are expressed as the percentage of stimulation related to the value of internalisation obtained without addition of any compound. The same method is used for the measurement of LDL internalisation.

Flow Cytometry.

For analysis of the cell surface EGF receptor, HepG2 cells are preincubated in medium with or without 20 nM EGF for 10 min at 37° C. For flow cytometry analysis, HepG2 and CHO cells are detached by incubation with Ca2+, Mg2+free PBS containing 2 mM EDTA, pH 7.4, fixed in 3% paraformaldehyde and pelleted in a microfuge. Cells are incubated at 20° C. for 1 h in PBS, pH 7.4 containing 1% BSA with either mouse monoclonal antibodies against the human β-subunit of ATP synthase, the human cytochrome oxidase subunit I, the human EGF receptor or against isotypic control IgG. Cells are washed in PBS/BSA 1% and incubated at 20° C. for 30 min with goat anti-rabbit IgG conjugated to fluorescein isothiocyanate. After a final wash, cells are pelleted and resuspended in PBS/BSA 1% at a density of 1×106 cells/ml. The mean relative fluorescence after excitation at a wavelength of 488 nm is determined for each sample on a Coulter XL 4C flow cytometer and analysed with CELLQUEST software (Becton-Dickenson).

Immunofluorescence and Confocal Microscopy.

IHH and CHO cells are plated at 5×105 cells/ml on glass coverslips and allowed to adhere overnight. Cells are washed with PBS, pH 7.4, fixed for 15 min in 3% paraformaldehyde and saturated for 30 min with 0.2% gelatin (staining buffer). A control slide is permeabilised for 2 min in 0.2% Triton X100. Cells are then incubated for 1 h with the primary antibody diluted (5 μg/ml) in PBS (mouse monoclonal IgG2a anti β-subunit of ATP synthase, mouse monoclonal IgG2a anti subunit I of cytochrome oxidase monoclonal or mouse IgG2a isotypic control). Immunostaining is performed for 1 h in the dark with anti-mouse alexa 488-conjugated IgG2a (5 μg/ml) in staining buffer. For confocal microscopy, cells are incubated for 2 h with 100 μg/ml apo AI, then washed 2 times in PBS before fixation. Rabbit polyclonal anti apoA-I immunserum (10 μg/ml) is co-incubated with primary antibodies as described above. Immunostaining is performed with anti-mouse alexa 488-conjugated IgG2a (5 μg/ml) and rhodamine-conjugated anti-rabbit IgG (5 μg/ml). The coverslips are examined with a Zeiss Axioskop microscope or with a confocal microscope (LSM510, Zeiss) at X630.

Cell Surface ADP and ATP Measurement.

Confluent HepG2 in 6-well plates are washed in DMEM, then incubated at 37° C. for 10 min in DMEM pH 6.6 with 0.1 μCi [α-32P] ATP for ADP generation assay, or with 0.1 μCi 32Pi and ADP (100 nM final) for ATP generation assay. Depending on the experiment, IF₁ (100 nM final) or apoA-I (10 μg/ml final) are added in the reaction mixture. Supernatants are removed and analysed using two different systems: 1) by HPLC coupled to a radioactivity detector on a Whatman Partisphere 5 SAX column (Whatman International Ltd., UK) as described previously (13); calibration is done with radiolabelled nucleotides. 2) by thin layer chromatography in the solvent NaCl 2.4%/NH4OH/H2O/MeOH (12.5/15/27.5/50, v/v); radioactive spots are counted by liquid scintillation.

### Example 1

Purification and Identification of a High-Affinity HDL Receptor on Hepatocytes.

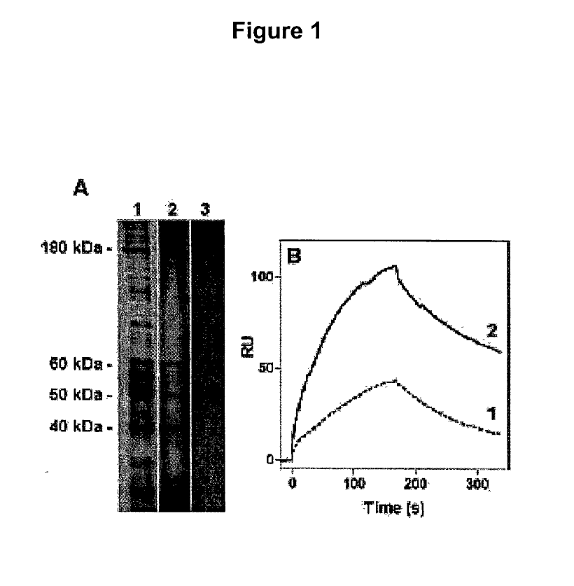

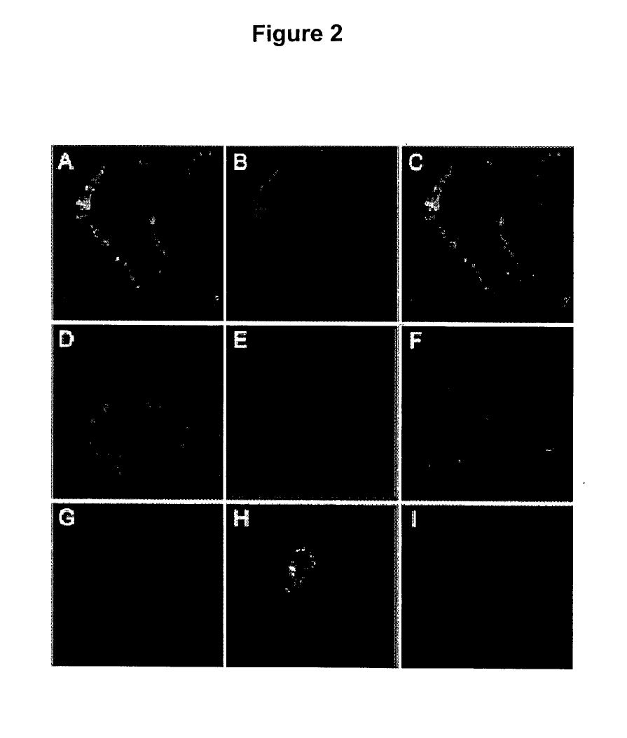

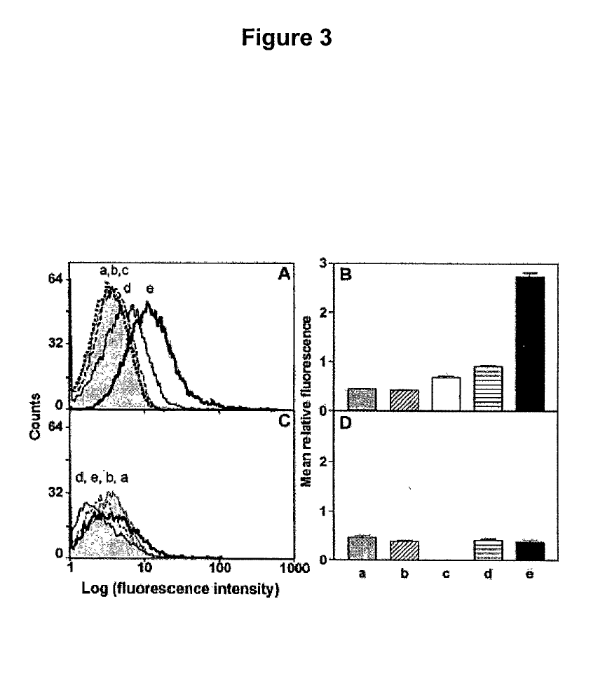

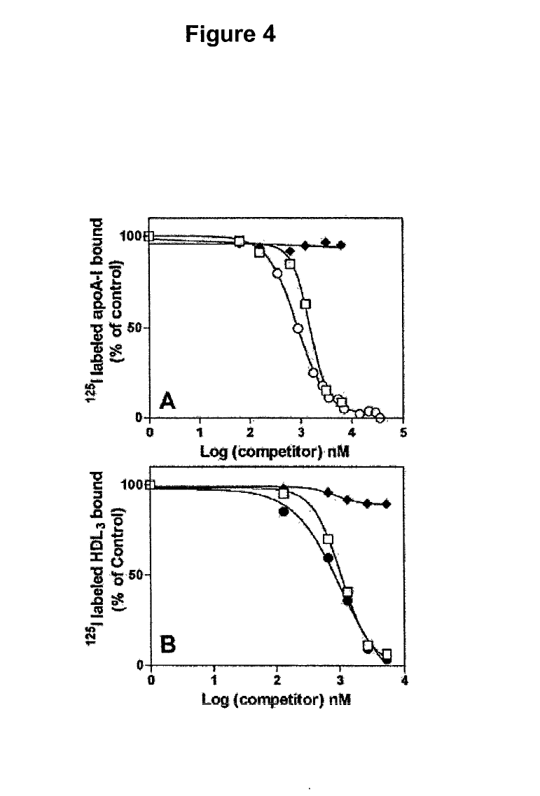

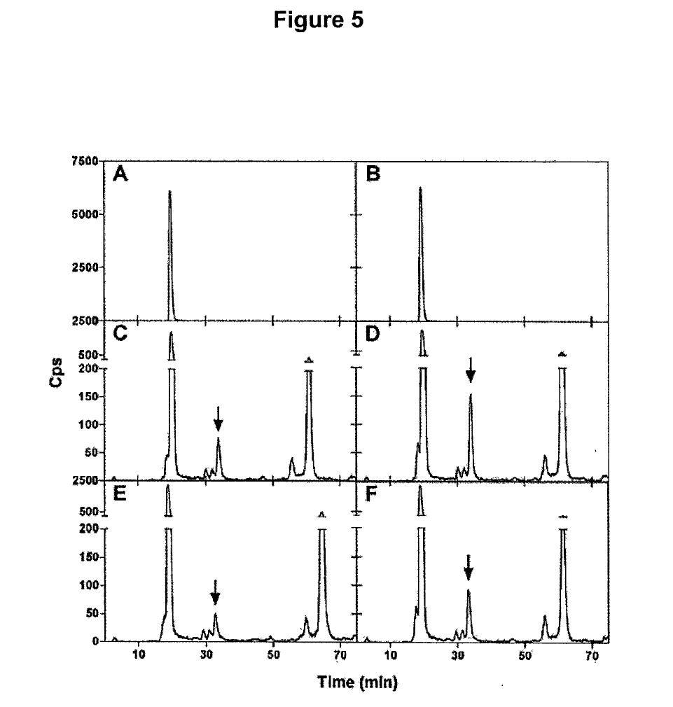

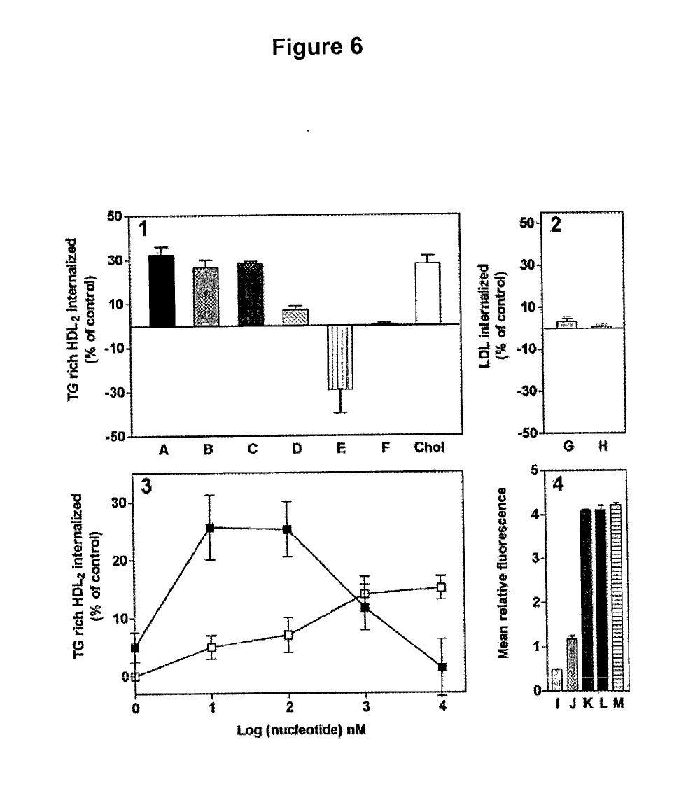

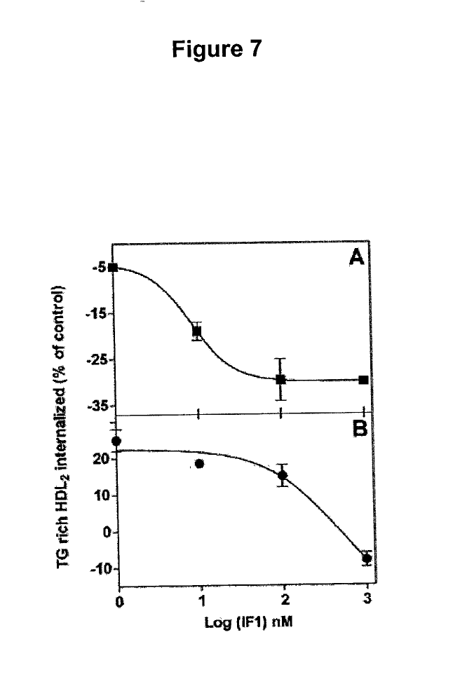

Immobilised free-apoA-I is used as a ligand and solubilised porcine liver plasma membrane proteins is used as starting material. First, surface plasmon resonance (Biacore) experiments indicate that interactions between solubilised porcine liver plasma membrane proteins and immobilized free-apoA-I are conserved with a high affinity dissociation constant (Kd^{˜}10-9 M, FIG. 1B, sensogram 1). Using multiple rounds of binding-desorption of solubilised membrane proteins on the sensor chip, a high concentration of apo-AI affinity bound proteins (2.5 ng/μl) are recovered. Using SDS/PAGE a 50 kDa protein (FIG. 1A, lane 3) is identified.

### Example 2

Improving the Recovery of the p50 Protein.

To improve the recovery of the p50 protein, free-apoA-I is immobilised on an affinity chromatography column (affigel 15—Bio-Rad), and using the same elution conditions as for the Biacore experiments, 4 main proteins are identified (FIG. 1A, lane 2) including the p50 in sufficient amount (50 pmole) to micro-sequence it. The eluted material is able to bind immobilised free-apoA-I (FIG. 1B, sensogram 2) with a relative increase of 4 fold as compared to crude solubilised homogenate (sensogram 1). Micro-sequencing is performed after protease digestion of the sliced-gel protein, HPLC separation and analysis by the Edman method. One peptide sequence derived from p50 is identical with a segment of the human β-subunit of ATP synthase.

### Example 3

Cell Surface Localisation of the β-Subunit of ATP Synthase and Binding of Free-apoA-I.

To demonstrate that the β-subunit of ATP synthase is present on the hepatocyte cell surface, immunofluorescence microscopy is used with an anti β-subunit monoclonal antibody. The presence of the β-subunit of ATP synthase on the cell surface of Immortalised Human Hepatocytes (IHH) is confirmed (FIG. 2B). This protein is absent on the CHO cell surface (FIG. 2C), which correlates well with the absence of high-affinity binding sites in this cell line (data not shown). By contrast, a strong intracellular fluorescence is observed on permeabilised IHH (FIG. 2A) or CHO cells (not shown), reflecting the ATP synthase present in mitochondria. As a negative control, isotypic mouse IgG displayed no fluorescent signal indicating the specificity of the ATP synthase IgG response (FIGS. 2D, E, F). Following preincubation of cells with apoA-I, confocal immunofluorescence microscopy with rabbit anti apoA-I is used detect a specific cell surface signal (FIG. 2H), that is strictly superimposed (FIG. 2I) to theβ subunit of ATP synthase (FIG. 2G). This confirms the colocalisation of the β-subunit of ATP synthase and apo AI binding at the hepatocyte cell surface. To further ascertain this localisation, human hepatoma cells (HepG2) are analysed by fluorescence-assisted flow cytometry using the anti βsubunit monoclonal antibody (FIGS. 3A, B). Experiments are also performed using CHO cells as a negative control (FIGS. 3C, D). In intact HepG2 cells (selected as the cell excluding propidium iodine) the presence on the cell surface of the β-subunit of ATP synthase is confirmed but not in CHO cells (FIG. 3, curve b). The selectivity of the response to β-subunit of ATP synthase is clearly demonstrated by the much lower signal obtained with either isotypic IgG (FIG. 3, curve a) or with a monoclonal antibody raised against another typical mitochondrial protein, the subunit I of cytochrome oxidase (FIG. 3, curve d). At the cell surface the presence of the β-subunit of ATP synthase is revealed by both immunofluorescence microscopy and by flow cytometry using monoclonal anti-β-subunit of ATP synthase (data not shown). This strongly suggests that, at least, the whole F₁-ATPase domain is present at the cell surface of the hepatocytes. Finally, incubation of hepatocytes with an excess of free-apoA-I almost completely abolishes (more than 3 fold) the immunoreactivity of the anti β-subunit antibody, confirming the cell surface interaction of free-apoA-I with β-subunit of ATP synthase (curve c in FIGS. 3A, B).

### Example 4

Confirmation of the Association Between Free-apoA-I and β-subunit of ATP Synthase.

To definitely conclude to the association between free-apoA-I and β-subunit of ATP synthase, two types of competition experiments are performed: first when ¹²⁵I-labeled free-apoA-I is used on HepG2 cells, anti β-subunit of ATP synthase monoclonal antibody (FIG. 4A) as well as anti β-subunit Fab fragments (not shown) completely inhibits the binding of ¹²⁵I-labeled free-apoA-I (at the same level as the unlabelled ligand itself). Secondly, HDL3 is used, which is one of the most abundant HDL sub-particles, already described to bind both the high and low affinity binding sites through their apoA-I (2). When ¹²⁵I-labeled HDL3 are used on inverted purified mitochondria, thus exposing outside the F₁ fraction of ATP synthase, but avoiding interferences with the cell surface low-affinity HDL binding sites (FIG. 4B), again, anti β-subunit of ATP synthase monoclonal antibody or Fab fragments (not shown) inhibit the binding of ¹²⁵I-labeled HDL3. In both cases, non-relevant antibodies have no inhibitory effect.

### Example 5

ATPase Activity Associated with the Cell Surface β-subunit of ATP Synthase.

To check the functional activity of cell surface ATP synthase, HepG2 cells are incubated with either ADP plus 32Pi, to detect ATP synthesis activity, or with [α-³²P]-ATP, to measure ATP hydrolytic activity. The different nucleotides generated in cell culture medium are identified by both Thin Layer Chromatography (TLC) and HPLC techniques, the latter allowing the precise quantification of the nucleotides. When ADP plus 32Pi are incubated for 10 minutes in the absence (FIG. 5A) or in the presence of free-apoA-I (FIG. 5B), no synthesis of ATP is detected by either HPLC or TLC (not shown). By contrast, incubation of the cells for 10 minutes at 37° C. with [α-32P]-ATP generates [α32P]-ADP (FIG. 5C, arrow) which is dramatically increased, up to 79%, in the presence of apoA-I (FIG. 5D), suggesting that binding of free-apoA-I to the β-subunit of ATP synthase stimulates the hydrolysis of ATP to ADP. To confirm this later observation, the purified IF₁ protein is used, the natural inhibitor protein of mitochondrial F₁-ATPase, which interacts with the P-subunit to inhibit the hydrolytic activity of the ATP synthase (14). When the cells are incubated for 10 minutes at 37° C., a strong decrease of the [α-32P]-ADP generated is observed (FIG. 5E, showing a 48% decrease as compared to control in FIG. 5C). Moreover, IF₁ protein could inhibit the stimulatory effect of free-apoA-I on ATP hydrolysis (FIG. 5F). Altogether, our data strongly suggests that the ATP synthase present on the plasma membrane of hepatocytes functions as an ATP hydrolase, and can be stimulated by free-apoA-I.

The presence on the cell surface of hepatocytes of both the α- and β-subunits of ATP synthase, strongly suggests that the entire F₁-ATPase can be present. It is well established that the ATP synthesis activity is dependent on an electrochemical proton gradient induced through the Fo sub-unit. In the absence of this gradient, ATP synthase turns to hydrolyse ATP to ADP. Interestingly, the IF₁ protein, which inhibits only the ATP hydrolysis activity of the F₁-ATPase, induces a decrease of the ADP present in the cell medium, demonstrating that the measured ATP hydrolysis is dependent on F₁-ATPase. The presence of extracellular ATP, physiologically or in culture medium, is well documented (15). Also, different ATP or ADP hydrolysis activities are described at the cell surface, and some phosphatases like members of the ecto-ATPase family have been identified (16); However, the effect of extracellular phosphatases in our ADP measurement can be excluded at least in the time course of our experiments. In addition to the specific inhibitory effect of IF, protein on ATP hydrolysis, these phosphatases should hydrolyse ATP to AMP, and the TLC experiments clearly show the almost complete absence of AMP (<5% of total radioactivity, not shown).

### Example 6

Specificity of the Nucleotides Effects Towards Triglyceride Rich-HDL2 Internalisation by Hepatocytes.

A particular subclass of HDL, the triglyceride rich-HDL2 particles, formed through remodelling of HDL2 by the lipid transfer proteins, binds only to the low affinity binding sites. By contrast, remnant-HDL2, a particle generated following action of hepatic lipase on triglyceride rich-HDL2, is able to bind to both the low and high affinity binding sites and is faster internalised and in higher amounts than the native triglyceride rich-HDL2 (4). These observations suggest that the ability to bind to high affinity HDL binding sites might stimulate the internalisation of HDL through the low affinity binding sites. In a preliminary experiment, when adding increasing concentrations of free-apoA-I (from 0.1 to 20 μg/ml) to HepG2 or IHH cells in the presence of ¹²⁵I-labeled TGHDL 2, the radioactivity internalised into the cells is increased (not shown). The maximum of stimulation was obtained between 5 to 15 min of incubation.

To assess the influence of the βsubunit of ATP synthase as an apoA-I receptor, on the internalisation of HDL into hepatocytes, the internalisation of ¹²⁵I-TG-HDL2 on HepG2 cells is measured, in the presence of ADP which, as described above, is the only compound produced by the ATP synthase complex at the hepatocyte cell surface. 100 nM ADP stimulates the internalisation of TG-HDL2 (FIG. 6-1B) at a level comparable to that observed with free-apoA-I (FIG. 6-1A). Addition of both ADP and free-apoA-I does not further increase the stimulation level (FIG. 6-1C), suggesting that the effects of both effectors addresses a similar endocytotic pathway. By contrast, a similar concentration of ATP only induces a small stimulation of TG-HDL2 internalisation (FIG. 6-1D). Moreover, apyrase (E.C. 3.6.1.5), which hydrolyses both ATP and ADP, completely abolishes the basal or the apoA-I stimulated endocytosis of TG-HDL2 (FIG. 6-1E), strengthening the presence of an ADP specific dependent pathway. Dose-response experiments with non-hydrolyzable nucleotides (FIG. 6-3) shows a stimulatory effect of 2MeS-ADP, a non hydrolizable analog of ADP, at 10 to 100 nM, followed by an inhibition of endocytosis at higher concentrations (1 μM to 10 μM). By contrast, ATPγS has a weak effect at low concentrations, slightly increasing (15% stimulation) endocytosis at a very high concentration (10 μM). These data strongly suggest that HDL endocytosis is ADP-dependent. Finally, addition of EGF (which is known to induce endocytosis of the EGF receptor) does not stimulate HDL internalisation (FIG. 6-1F), indicating that HDL processing is not dependent on a non specific general activation of endocytosis. When experiments are performed with 3Hcholesteryl ether-labeled-TG-HDL2 (FIG. 6-1 Chol), the level of stimulation of internalisation by free-apo AI is similar to that of the protein moiety (FIG. 6-1A), indicating that the holoparticle HDL is implicated in the endocytic process. It has been recently proposed that HDL endocytosis could occur by two different pathways in hepatocytes: one is dependent on Scavenger receptor class B type I (SR-BI), a wildly described HDL receptor (17), internalisation with the holo HDL particles and represents a selective transcytosis of lipoprotein cholesterol, which could explain the selective sorting of cholesterol to the bile canaliculus; the other, independent of SR-BI, could involve the uptake and degradation of the holo-HDL particle by unknown receptors (18). Thus endocytosis of HDL could be the primary event for both pathways.

### Example 7

Stimulation of Endocytosis by Free-apoA-I or ADP is Specific Towards HDL.

When ¹²⁵I-labeled LDL is used, no stimulation of endocytosis is observed with either apoA-I or ADP (FIGS. 6-2G and 6-2H respectively). Moreover, measurement of EGF receptor (EGF-R) internalisation, as a tyrosine kinase type receptor, by flow cytometry (FIG. 6-4) indicates that while EGF strongly reduces the number of EGF-R at the cell surface (FIG. 6-4J), when compared to control cells (FIG. 6-4K), neither free apoA-I (FIG. 6-4L) nor ADP (FIG. 6-4M) are able to reduce the presence of EGF-R on HepG2 cells. These experiments are repeated on IHH cells giving similar results (not shown).

### Example 8

Effect of IF₁ Protein on TG-HDL2 Internalisation

IF₁ protein is a natural inhibitor of the ATP hydrolysis activity of mitochondrial ATP synthase (7). When internalisation of TG-HDL2 is measured in the presence of increasing concentrations of IF₁, a dramatic decrease of HDL endocytosis is observed (FIG. 7A). Furthermore, higher concentrations of IF₁ protein are also able to completely abolish the stimulation of the TG-HDL2 endocytosis by free-apoA-I (FIG. 7B). Altogether, our data lead us to propose a mechanism whereby the ectopic ATP synthase present on the surface of hepatocyte, hydrolyses extracellular ATP to ADP, which in turn activates the HDL endocytosis. This mechanism is stimulated by the high affinity binding of apoA-I to the β-chain of ATP synthase, inducing an overproduction of ADP and increasing HDL endocytosis. Although we already suggested above that ABCA-I was not involved in the binding of apoA-I, to conclude to the absence of contribution of ABCA-1 in our observations, we measured the influence of ADP and IF1 protein on cholesterol efflux, a typical feature of ABCA-1. Using HepG2 cells and IHH we showed, as previously described 16, a stimulation (2 to 2.5 fold) of the cholesterol efflux induced by free-apoA-I. Nevertheless, no influence of IF1 or ADP (in a range of 1 nM to 10 μM) was detectable, allowing us to conclude that ABCA-I was not implicated in our observations.

Finally, to estimate the physiological relevance of the data, we performed in situ experiments using perfused rat liver (Table 1). Interestingly, TG-HDL2 internalisation by the liver was quickly (45 min) and dramatically decreased (up to 45%) in the presence of IF1 protein, indicating that in rodent the ectopic ATP synthase seems to be implicated in hepatic HDL endocytosis.

### Example 9

Assay Method for Identifying Agents that Modulate Lipoprotein Endocytosis.

Confluent HepG2 in 6-well plates are washed in DMEM, then incubated at 37° C. for 10 min in DMEM pH 6.6 with 0.1 μCi [α-32P] ATP for ADP generation assay. An agent is added to one reaction mixture and a control without an agent is also used. Supernatants are removed and analysed using two different systems: 1) by HPLC coupled to a radioactivity detector on a Whatman Partisphere 5 SAX column (Whatman International Ltd., UK) as described previously (13); calibration is done with radiolabelled nucleotides. 2) by thin layer chromatography in the solvent NaCl 2.4%/NH40H/H20/MeOH (12.5/15/27.5/50, v/v); radioactive spots are counted by liquid scintillation.

If the agent increases the amount of ADP generation in comparison to the control reaction mixture then modulation of lipoprotein endocytosis is determined.

Cells are washed three times and pre-incubated for 30 min at 37° C. in serum free DMEM. ¹²⁵I triglyceride rich-HDL2 (75 μg/ml) is added on the cells and incubated at 37° C. for various times. Radiolabelled lipoprotein may be prepared as previously described. Free apolipoprotein A-I (10 μg/ml) is added to the incubation medium as well as the agent and a control without the agent is also used. At each incubation time, cells are washed with cold DMEM and the release of radioactive ligands associated to the cell surface is performed at 4° C., for 90 min in DMEM. The radioactivity internalised into the cells and the protein content is determined as in the competition assays as previously described. Non-specific internalisation is analysed in the presence of an excess of 600 μg/ml of HDL3. Results are expressed as the percentage of stimulation related to the value of internalisation obtained with and without the addition of the agent.

CONCLUSIONS

The experimental data described herein demonstrates the presence of the β-subunit, probably associated with its counterpart, the α-subunit, on the cell surface of HepG2, IHH or primary human hepatocytes, but not on epithelial cells like CHO. This observation suggests that the presence at the cell surface of this protein is more dependent on the cell type than on the tumorigenic status of the cells as suggested by Das et al (10). Thus, the F₁ domain of the ATP synthase of the lipoprotein receptor of the present invention may comprise the β-subunit associated with the α-subunit.

In the present study, apoA-I high-affinity binding sites on hepatocytes are purified and characterised and identified as the β-subunit of human ATP synthase, a major protein complex of mitochondria inner membrane, involved in ATP synthesis. Mitochondrial ATP synthase has two major domains, F₁ and Fo (5). F₁ is a peripheral membrane protein complex, which consists of five different subunits (among them, the β-subunit), containing binding sites for ATP and ADP, including the catalytic site for ATP synthesis. F₁ is held to the membrane by its interaction with Fo, an integral membrane protein complex in mammalian mitochondria that contains a transmembrane channel through which protons can cross the membrane (6). The synthesis of ATP requires an electrochemical proton gradient across the inner mitochondrial membrane. The collapse or the absence (for instance, when the F₁ complex is present alone) of the electrochemical proton gradient induces a switch of the enzymatic activity from ATP synthesis to ATP hydrolysis. In this case, the catalytic domain of ATP synthase, present mainly in the β-subunits, catalyzes the hydrolysis of ATP to ADP and phosphate, an activity that is regulated in mitochondria by a natural inhibitor protein, IF₁ (7, 8)]. The role of the β-subunit of ATP synthase on HDL metabolism has been elucidated. The β-subunit of ATP synthase is the high affinity apoA-I receptor, present on the cell surface of hepatocyte. Using the high specificity of IF₁ for the ATP synthase, we also show a strict relationship between its ATP hydrolysis activity, stimulated by free-apoA-I, and the HDL endocytosis. For the first time, a role for cell surface ATP synthase in the hepatic uptake of HDL is assigned, the last step in reverse cholesterol transport.

Without being bound by any particular theory, this data is indicative of a mechanism whereby the ectopic ATP synthase present on the surface of hepatocytes, hydrolyses extracellular ATP to ADP, which in turn activates HDL endocytosis. This mechanism is stimulated by the high affinity binding of apoA-I to the β-subunit of ATP synthase, inducing an overproduction of ADP and increasing HDL endocytosis. The proposed role for cell surface ATP synthase in HDL catabolism opens new perspectives in the control of cholesterolemia, which is a major issue in cardiovascular disease research. However, questions as how the cell processes those proteins towards the cell surface, or the regulation of its expression (which seems to be restricted to certain cell types) remain unknown and require further investigation. Nevertheless, the increasing number of publications describing mitochondrial proteins present at the cell surface and the comprehension of this phenomenon opens a new field of investigation.

Other aspects of the present invention relate to an assay method comprising the steps of: (a) identifying a lipoprotein receptor according to the method of the present invention; (b) determining if said lipoprotein receptor modulates lipoprotein endocytosis; and (c) identifying one or more agents that affect the modulation of lipoprotein endocytosis.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1
TG-HDL2 internalisation by perfused rat liver

| Control | IF1 |
|---|---|
| (μgTG-HDL2 /g of liver) | (μgTG-HDL2/g of liver) |
| 5.904 ± 1.902 | 2.597 ± 1.026 |

The liver from male Whistar rats (7-8 weeks old) were perfused in situ during 45 minutes at 37° C. in Ringer medium (pH 6.8) with 35 μg/ml of 1251-TG-HDL2 with or without 1 μM IF1. Livers were then extensively washed at 4° C. and radioactivity counted. n=7 per group. Values represent means±SEM, with p<0.02 (unpaired t-test). Control fluorescence-assisted flow cytometry experiments have shown the presence of both the Band a-chain ATP synthase at the cell surface of isolated rat hepatocytes (not shown).

### REFERENCES

1. J. R. Morrison, G. A. McPherson, N. H. Fidge, J. Biol. Chem. 267, 13205-13209 (1992).

2. R. Barbaras, X. Collet, H. Chap, B. Perret, Biochemistry 33, 2335-2340 (1994).

3. A. Garcia et al., Biochemistry 35, 13064-13070 (1996).

4. K. Guendouzi, X. Collet, B. Perret, H. Chap, R. Barbaras, Biochemistry 37, 14974-14980 (1998).

5. P. D. Boyer, Annu Rev Biochem 66, 717-49 (1997).

6. D. Stock, A. G. Leslie, J. E. Walker, Science 286, 1700-5. (1999).

7. D. A. Harris, A. M. Das, Biochem J 280, 561-73. (1991); E. Cabezon, M. J. Runswick, G. Leslie, J. E. Walker, Embo J 20, 6990-6. (2001).

8. J. E. Walker, Curr Opin Struct Biol 4, 912-8. (1994).

9. L. O. Martinez et al., Biochemistry 39, 1076-1082 (2000).

10. B. Das, M. O. Mondragon, M. Sadeghian, V. B. Hatcher, A. J. Norin, J Exp Med 180, 273-81. (1994).

11. T. L. Moser et al., Proc Natl Acad Sci U S A 96, 2811-6. (1999); S. Y. Chang, S. G. Park, S. Kim, C. Y. Kang, J Biol Chem 11, 11 (2001).

12. T. L. Moser et al., Proc Natl Acad Sci U S A 98, 6656-61. (2001).

13. U. Beisiegel et al., Arteriosclerosis 8, 288-97. (1988); R. W. Mahley, D. Y. Hui, T. L. Innerarity, U. Beisiegel, Arteriosclerosis 9, 114-8. (1989).

14. E. Cabezon, I. Arechaga, P. Jonathan, G. Butler, J. E. Walker, J Biol Chem 275, 28353-5. (2000); E. Cabezon, P. J. Butler, M. J. Runswick, J. E. Walker, J Biol Chem 275, 25460-4. (2000).

15. V. Ralevic, G. Burnstock, Pharmacol Rev 50, 413-92. (1998).

16. L. Plesner, Int Rev Cytol 158, 141-214 (1995). 14

17. S. Acton et al., Science 271, 518-20 (1996).

18. D. L. Silver, W. Nan, X. Xiao, A. R. Tall, J Biol Chem 276, 25287-25293 (2001).

19. Ross, R., Nature, 362, 801-809 (1993).

20. Barter & Rye (1996) Atherosclerosis 121:1-12

21. Lamarche & Lewis (1998) Can. J. Cardiol. 14:841-851

22. Lacko et al. (1997) J. Lipid Research 38:1267-1273

1. A method for identifying a lipoprotein receptor comprising the steps of:
contacting a sample with lipoprotein;
obtaining one or more lipoprotein bound proteins; and
determining if the lipoprotein bound proteins comprise at least one domain of ATP synthase.

2. The method according to claim 1 wherein the lipoprotein is high density lipoprotein (HDL).

3. The method according to claim 1 wherein the lipoprotein is lipid free-apolipoprotein A-I (free-apoA-I).

4. The method according to claim 1 wherein at least one domain of ATP synthase comprises one or more subunits of the F₁ domain of ATP synthase.

5. The method according to claim 4 wherein at least one domain of ATP synthase comprises the beta-subunit of the F₁ domain of ATP synthase.

6. The method according to claim 5 wherein the beta-subunit of the F₁ domain of ATP synthase comprises SEQ ID No.1.

7. The method according to claim 5 wherein the beta-subunit of the F₁ domain of ATP synthase comprises a polypeptide encoded by SEQ ID No.2.

8. The method according to claim 1 wherein the sample comprises solubilised membranes.

9. The method according to claim 8 wherein the solubilised membranes are solubilised liver plasma membranes.

10. The method according to claim 1 wherein the sample is contacted with immobilised lipoprotein.

11. The method according to claim 10 wherein the sample is contacted with immobilised lipoprotein using surface plasmon resonance or affinity chromatography.

12. The method according to claim 1 comprising the additional step (d) of determining if at least one domain of ATP synthase is localised at the surface of a cell.

13. The method according to claim 12 wherein step (d) is performed using immunofluorescence microscopy or fluorescence assisted flow cytometry.

14. The method according to claim 13 wherein immunofluorescence microscopy or fluorescence assisted flow cytometry is performed with an anti-ATP synthase monoclonal antibody.

15. The method according to claim 14 wherein the antibody is an anti-β-subunit ATP synthase monoclonal antibody.

16. An assay method comprising the steps of:
identifying one or more agents that modulate ATP hydrolysis; and
determining if the one or more agents modulate the activity of a lipoprotein receptor.

17. The assay method according to claim 16 wherein the lipoprotein receptor modulates lipoprotein endocytosis.

18. The assay method according to claim 16 wherein the lipoprotein receptor stimulates HDL endocytosis.

19. The assay method according to claim 16 wherein the lipoprotein receptor stimulates holo-HDL endocytosis.

20. The assay method according to claim 16 wherein ATP is hydrolysed by at least one domain of ATP synthase.

21. The assay method according to claim 20 wherein at least one domain of ATP synthase comprises one or more subunits of the F₁ domain of ATP synthase.

22. The assay method according to claim 20 wherein at least one domain of ATP synthase comprises the beta-subunit of the F₁ domain of ATP synthase.

23. The assay method according to claim 22 wherein the beta-subunit of the F₁ domain of ATP synthase comprises SEQ ID No.1.

24. The assay method according to claim 22 wherein the beta-subunit of the F₁ domain of ATP synthase comprises a polypeptide encoded by SEQ ID No.2.

25. The assay method according to claim 16 wherein at least one domain of ATP synthase is present on the surface of a cell.

26. The assay method according to claim 16 wherein the lipoprotein receptor is a high density lipoprotein (HDL) receptor.

27. The assay method according to claim 16 wherein the lipoprotein receptor is a lipid free-apolipoprotein A-I (free-apoA-I) receptor.

28. The assay method according to claim 16 wherein the agents modulate the activity of a further entity.

29. The assay method according to claim 16 wherein the agent is an antagonist that decreases lipoprotein endocytosis.

30. The assay method according to claim 16 wherein the agent is an agonist that increases lipoprotein endocytosis.

31. The assay method according to claim 30 wherein the agent is an antagonist of IF1.

32. The assay method according to claim 16 wherein the assay method is used to screen for agents that are useful in the treatment and/or prevention of disease.

33. A process comprising the steps of:
performing the assay method according to claim 16;
identifying an agent capable of modulating lipoprotein endocytosis; and
preparing a quantity of that agent.

34. A process comprising the steps of:
performing the assay according to claim 16;
identifying an agent capable of modulating lipoprotein endocytosis;
preparing a quantity of that agent; and
preparing a pharmaceutical composition comprising that agent.

35. A process comprising the steps of:
performing the assay according to claim 16;
identifying an agent capable of modulating lipoprotein endocytosis;
modifying said agent; and
preparing a pharmaceutical composition comprising said modified agent.

36. A pharmaceutical composition comprising an agent identified by the assay method of claim 16 or the process of any one of claims 48 to 50 admixed with a pharmaceutically acceptable carrier, diluent, excipient or adjuvant and/or combinations thereof.

37. A process of preparing a pharmaceutical composition comprising admixing an agent identified by the assay method of claim 16 or the process of any one of claims 48 to 50 with a pharmaceutically acceptable diluent, carrier, excipient or adjuvant and/or combinations thereof.

38. A method of treating a disease in a human or animal which method comprises administering to an individual an effective amount of a pharmaceutical composition comprising an agent identified by the assay method of claim 16 or the process of any one of claims 48 to 50, wherein the agent is capable of modulating the disease and wherein said composition is optionally admixed with a pharmaceutically acceptable carrier, diluent excipient or adjuvant and/or combinations thereof.

39. The method according to claim 38 wherein said one or more agents are formulated into one or more compositions for use in medicine.

40. An agent identified by the assay method according to claim 16.

41. The assay method according to claim 32, wherein the disease is selected from: cardiovascular disease, coronary heart disease, stroke, pancreatitis, atherosclerosis, gout, and/or type 2 diabetes.

42. The method according to claim 38, wherein the disease is selected from: cardiovascular disease, coronary heart disease, stroke, pancreatitis, atherosclerosis, gout, and/or type 2 diabetes.


SEQUENCE LISTING (2 sequences)

SEQ ID NO: 1 - protein, 539 aa, Homo sapiens
MTSLWGKGTGCKLFKFRVAAAPASGALRRLTPSASLPPAQLLLRAVRRRSHPVRDYAAQTSPSPKAGAATGRIVAVIGAVVDVQFDEGLPPILNALEVQGRETRLVLEVAQHLGESTVRTIAMDGTEGLVRGQKVLDSGAPIKIPVGPETLGRIMNVIGEPIDERGPIKTKQFAPIHAEAPEFMEMSVEQEILVTGIKVVDLLAPYAKGGKIGLFGGAGVGKTVLIMELINNVAKAHGGYSVFAGVGERTREGNDLYHEMIESGVINLKDATSKVALVYGQMNQPPGARARVALTGLTVAEYFRDQEGQDVLLFIDNIFRFTQAGSEVSALLGRIPSAVGYQPTLATDMGTMQERITTTKKGSITSVQAIYVPADDLTDPAPATTFAHLDATTVLSRAIAELGIYPAVDPLDSTSRIMDPNIVGSEHYDVARGVQKILQDYKSLQDIIAILGMDELSEEDKLTVSRARKIQRFLSQPFQVAEVFTGHMGKLVPLKETIKGFQQILAGEYDHLPEQAFYMVGPIEEAVAKADKLAEEHSS

SEQ ID NO: 2 - DNA, 1807 bp, Homo sapiens
gaattctttcttcagcccatgtaaacatgaaaataagggttaaaaatgacttcattatggggaaaagggacaggatgcaaattgttcaaattccgggtggccgctgctccggcctccggggccttgcggagactcaccccttcagcgtcgctgcccccagctcagctcttactgcgggccgtccgacggcggtcccatcctgtcagggactatgcggcgcaaacatctccttcgccaaaagcaggcgccgccaccgggcgcatcgtggcggtcattggcgcagtggtggacgtccagtttgatgagggactaccaccaattctaaatgccctggaagtgcaaggcagggagaccagactggttttggaggtggcccagcatttgggtgagagcacagtaaggactattgctatggatggtacagaaggcttggttagaggccagaaagtactggattctggtgcaccaatcaaaattcctgttggtcctgagactttgggcagaatcatgaatgtcattggagaacctattgatgaaagaggtcccatcaaaaccaaacaatttgctcccattcatgctgaggctccagagttcatggaaatgagtgttgagcaggaaattctggtgactggtatcaaggttgtcgatctgctagctccctatgccaagggtggcaaaattgggctttttggtggtgctggagttggcaagactgtactgatcatggagttaatcaacaatgtcgccaaagcccatggtggttactctgtgtttgctggtgttggtgagaggacccgtgaaggcaatgatttataccatgaaatgattgaatctggtgttatcaacttaaaagatgccacctctaaggtagcgctggtatatggtcaaatgaatcaaccacctggtgctcgtgcccgggtagctctgactgggctgactgtggctgaatacttcagagaccaagaaggtcaagatgtactgctatttattgataacatctttcgcttcacccaggctggttcagaggtgtctgcattattgggccgaatcccttctgctgtgggctatcagcctaccctggccactgacatgggcactatgcaggaaagaattaccactaccaagaagggatctatcacctctgtacaggctatctatgtgcctgctgatgacttgactgaccctgcccctgctactacgtttgcccatttggatgctaccactgtactgtcgcgtgccattgctgagctgggcatctatccagctgtggatcctctagactccacctctcgtatcatggatcccaacattgttggcagtgagcattacgatgttgcccgtggggtgcaaaagatcctgcaggactacaaatccctccaggatatcattgccatcctgggtatggatgaactttctgaggaagacaagttgaccgtgtcccgtgcacggaaaatacagcgtttcttgtctcagccattccaggttgctgaggtcttcacaggtcatatggggaagctggtacccctgaaggagaccatcaaaggattccagcagattttggcaggtgaatatgaccatctcccagaacaggccttctatatggtgggacccattgaagaagctgtggcaaaagctgataagctggctgaagagcattcatcgtgaggggtctttgtcctctgtacttgtctctctccttgcccctaacccaaaaagcttcatttttctatataggctgcacaagagccttgattgaagatatattctttctgaacagtatttaaggtttccaataaaatcggaattc